SEQUENCE LISTING (20 sequences)

SEQ ID NO: 1 - protein, 16 aa, synthetic construct
CAISESWTGGSDTQYF

SEQ ID NO: 2 - protein, 16 aa, synthetic construct
CAISESWAGGTDTQYF

SEQ ID NO: 3 - protein, 16 aa, synthetic construct
CAISEGWTGNTDTQYF

SEQ ID NO: 4 - protein, 15 aa, synthetic construct
CIVRPNTGTASKLTF

SEQ ID NO: 5 - protein, 15 aa, synthetic construct
CIVRGNTGTASKLTF

SEQ ID NO: 6 - protein, 17 aa, synthetic construct
MEETAIWEQHTVTLHRA

SEQ ID NO: 7 - protein, 9 aa, synthetic construct
TAIWEQHTV

SEQ ID NO: 8 - protein, 15 aa, synthetic construct
GGGIALWESHDVGGG

SEQ ID NO: 9 - protein, 9 aa, synthetic construct
IALWESHDV

SEQ ID NO: 10 - protein, 15 aa, synthetic construct
YGLVAIKEAHDIETR

SEQ ID NO: 11 - protein, 9 aa, synthetic construct
VAIKEAHDI

SEQ ID NO: 12 - protein, 15 aa, synthetic construct
TPYIGLAESHDNVCD

SEQ ID NO: 13 - protein, 9 aa, synthetic construct
IGLAESHDN

SEQ ID NO: 14 - protein, 15 aa, synthetic construct
MEELIWEQYTVTLQK

SEQ ID NO: 15 - protein, 9 aa, synthetic construct
ELIWEQYTV

SEQ ID NO: 16 - protein, 15 aa, synthetic construct
MEELTIWEQHTATLS

SEQ ID NO: 17 - protein, 9 aa, synthetic construct
LTIWEQHTA

SEQ ID NO: 18 - protein, 15 aa, synthetic construct
IPILAVMESHAIQVN

SEQ ID NO: 19 - protein, 9 aa, synthetic construct
LAVMESHAI

SEQ ID NO: 20 - DNA, 287 bp, Homo sapiens
  source 1..287: mol_type = genomic DNA
gatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagtc

(19) United States
(12) Patent Application Publication
(10) Pub. No.: US 2025/0074966 A1
(43) Pub. Date: Mar. 6, 2025

(54) AGENTS AND METHODS FOR PREVENTING OR INHIBITING ADAPTIVE IMMUNE RESPONSES ASSOCIATED WITH MULTIPLE SCLEROSIS

(71) Applicant: Adaptive Biotechnologies Corporation, Seattle, WA (US)

(72) Inventors: Mark Klinger, Seattle, WA (US); Amanda Moore, Seattle, WA (US); Rebecca Harris, Seattle, WA (US); Harlan S. Robins, Seattle, WA (US)

(21) Appl. No.: 18/822,195

(22) Filed: Aug. 31, 2024

Related U.S. Application Data
(60) Provisional application No. 63/535,750, filed on Aug. 31, 2023.

(51) Int. Cl. C07K 14/74; A61K 39/00; C07K 14/725; C07K 16/28; C12N 5/0783; C12N 15/113

(52) U.S. Cl. CPC C07K 14/70539; A61K 40/11; A61K 40/15; A61K 40/31; A61K 40/32; A61K 40/416; C07K 14/7051; C07K 16/2809; C12N 5/0637; C12N 15/1138; C07K 2319/00; C12N 2310/11; C12N 2310/14; C12N 2510/00

(57) ABSTRACT

Provided are agents and methods for preventing or inhibiting a multiple sclerosis (MS)-associated adaptive immune response in a subject in need thereof. In some instances, the agents comprise or consist of a soluble peptide-major histocompatibility complex (pMHC) or multimer(s) thereof. The peptide of the pMHC is one identified as being targeted by MS-associated T-cell receptors (TCRs). The agents may be used alone or may be conjugated or fused to one or more moieties that result in the targeted depletion of T cells expressing the MS-associated TCRs. Other agents such as the MS-associated TCRs or binding domains thereof, and inhibitory immune cells expressing such TCRs are also provided. Aspects of the present disclosure further include methods of treating multiple sclerosis in a subject in need thereof via administration of an agent of the present disclosure.

### CROSS-REFERENCE TO RELATED APPLICATIONS

This application claims the benefit of U.S. Provisional Patent Application No. 63/535,750, filed Aug. 31, 2023, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “ADBS-097WO_SEQLIST”, created on Aug. 30, 2024 and having a size of 18,728 bytes. The contents of the Sequence Listing XML are incorporated herein by reference in their entirety.

### INTRODUCTION

Multiple sclerosis (MS) is an immune-mediated inflammatory disease that attacks myelinated axons in the central nervous system, destroying the myelin and the axon in variable degrees and producing significant physical disability within 20-25 years in more than 30% of patients. The hallmark of MS is symptomatic episodes that occur months or years apart and affect different anatomic locations.

Symptoms of MS include sensory loss (i.e., paresthesias), spinal cord symptoms (motor—e.g., muscle cramping secondary to spasticity), spinal cord symptoms (autonomic—e.g., bladder, bowel, and sexual dysfunction), cerebellar symptoms (e.g., Charcot triad of dysarthria (scanning speech), nystagmus, and intention tremor), optic neuritis, trigeminal neuralgia (e.g., bilateral facial weakness or trigeminal neuralgia), facial myokymia, diplopia on lateral gaze, and heat intolerance, among others.

Multiple sclerosis is an inflammatory, demyelinating disease of the CNS. In pathologic specimens, the demyelinating lesions of MS, called plaques (see the image below), appear as indurated areas—hence the term sclerosis. Examination of the demyelinating lesions in the spinal cord and brain of patients with MS shows myelin loss, destruction of oligodendrocytes, and reactive astrogliosis, often with relative sparing of the axon cylinder. In some MS patients, however, the axon is also aggressively destroyed.

The location of lesions in the CNS usually dictates the type of clinical deficit that results. As neural inflammation resolves in MS, some remyelination occurs, but some recovery of function that takes place in a patient could be due to nervous system plasticity. MS is also characterized by perivenular infiltration of lymphocytes and macrophages, as demonstrated in the image below. Infiltration of inflammatory cells occurs in the parenchyma of the brain, brainstem, optic nerves, and spinal cord.

One of the earliest steps in lesion formation is the breakdown of the blood-brain barrier. Enhanced expression of adhesion molecules on the surface of lymphocytes and macrophages is currently believed to underlie the ability of these inflammatory cells to penetrate the blood-brain barrier.

Molecular studies of white matter plaque tissue have shown that interleukin (IL)-12, a potent promoter of inflammation, is expressed at high levels in lesions that form early in MS. B7-1, a molecule required to stimulate lymphocytes to release proinflammatory cytokines, is also expressed at high levels in early MS plaques. Evidence exists of elevated frequencies of activated myelin-reactive T-cell clones in the circulation of patients with relapsing-remitting MS and higher IL-12 production in immune cells of patients with progressive MS.

Decreased function of T-lymphocytes with a regulatory role (Tregs) has been implicated in MS. These Tregs are CD4⁺ CD25⁺ T cells that can be identified by their expression of a transcription factor known as Foxp3.

Conversely, the cytokine IL-23 has been shown to drive cells to commit to a pathogenic phenotype in autoimmune diseases, including MS. These pathogenic CD4⁺ T cells act reciprocally to counteract Treg function and can be identified by their high expression of the proinflammatory cytokine IL-17; they are therefore referred to as T_{H} 17 cells.

Tregs and T_{H} 17 cells are not the only critical immune cells in the pathogenesis of MS. Immune cells such as microglia (resident macrophages of the CNS), dendritic cells, natural killer (NK) cells, and B cells are gaining increased attention by MS researchers. In addition, nonimmune cells (ie, endothelial cells) have also been implicated in mechanisms that lead to CNS inflammation.

MS is divided into the following categories, principally on the basis of clinical criteria, including the frequency of clinical relapses, time to disease progression, and lesion development on MRI: Relapsing-remitting MS (RRMS—approximately 85% of cases), secondary progressive MS (SPMS), primary progressive MS (PPMS), and progressive-relapsing MS (PRMS). Clinically isolated syndrome (CIS) and benign MS are sometimes included in RRMS.

Treatment of MS generally consists of immunomodulatory therapy (IMT) for the underlying immune disorder and therapies to relieve or modify symptoms. Methylprednisolone (Solu-Medrol) can hasten recovery from an acute exacerbation of MS. Plasma exchange (plasmapheresis) can be used short term for severe attacks if steroids are contraindicated or ineffective. Dexamethasone is commonly used for acute transverse myelitis and acute disseminated encephalitis. Most of the disease-modifying agents for MS (DMAMS) have been approved for use only in relapsing forms of MS. However, siponimod, ocrelizumab, ozanimod, and cladribine are also approved for active secondary progressive disease. The DMAMS currently approved for use by the US Food and Drug Administration (FDA) include interferons (e.g., IFN beta-1a, IFN beta-1b, peginterferon beta-1a), sphingosine 1-phosphate (S1P) receptor modulators (e.g., siponimod, fingolimod, ozanimod), monoclonal antibodies (e.g., natalizumab, alemtuzumab, ocrelizumab, ublituximab), and miscellaneous immunomodulators (e.g., glatiramer, mitoxantrone, teriflunomide, dimethyl fumarate, cladribine). A single-use autoinjector is also available for self-injection of interferon beta-1a (Rebif) in patients with relapsing forms of MS.

For treatment of aggressive MS, high-dose cyclophosphamide (Cytoxan) has been used for induction therapy, and mitoxantrone is approved for reducing neurologic disability and/or the frequency of clinical relapses in patients with SPMS, PRMS, or worsening RRMS.

Antigen-specific cellular immune responses are mediated by a diverse population of T cells and B cells, each bearing immune cell receptors (T cell receptors (TCRs) and B cell receptors (BCRs), respectively) capable of recognizing a specific antigen (in the case of T cells, an antigen peptide bound to a particular major histocompatibility complex (MHC) molecule on the surface of host cells). Encounter with an antigen leads to the clonal expansion, activation, and maturation of T and B cells, resulting in effector populations of cytotoxic (CD8⁺ CTL) and helper (CD4⁺) T cells, or antibodies and memory B cells, respectively. The presence of antigen-specific effector cells is diagnostic of an immune response specific to that antigen.

Helper T cells, according to their cytokine-secreting profile, have been divided into two major groups TH1 and TH2 cells. The former produce pro-inflammatory cytokines and lead effector immune mechanisms to be mainly or exclusively mediated by cytotoxic T and accessory cells such as NK cells and macrophages, while the latter produce cytokines that drive immunoglobulin production and isotype switch, leading to exuberant antibody mediated responses. The balance (or imbalance) of TH1 and TH2 cells determines the immunologic scenario and hence the clinical picture of immune mediated reactions or disease.

In the case of T cell mediated autoimmune disorders such as MS and Type 1 Diabetes (T1D), CD4+ T cells are key effectors of tissue damage. Disease susceptibility and/or resistance are strongly associated with certain MHC class II alleles.

The MHC system, known as the human leukocyte antigen (HLA) complex in humans, encodes proteins that make up MHC Class I, MHC Class II, or MHC Class III proteins. The HLA region, located on the short arm of chromosome 6 on band 6p21.3, is responsible for generating an immune response and is homologous to the MHC system. The MHC antigen presentation pathway is a critical process for the adaptive immune system that regulates T lymphocyte (T cell) immune responses toward pathogen-infected and cancerous cells. Antigen presentation occurs through MHC molecules, which present MAPs on the cell surface for recognition by T cells via T cell receptors. Through the recognition response, MHC molecules presenting MAPs bind to the T cell receptors to activate the T cell and initiate an immune response. The two major classes of MHC molecules are MHC Class I and II. Both MHC-I and -II are transmembrane cell surface molecules in the glycoprotein family.

The activation of a T cell is triggered by the binding of the TCR to antigen-derived peptides that are presented on major histocompatibility complex molecules (pMHCs). TCRs have an extensive sequence diversity, with estimates ranging from 1015 to 1061 different TCR sequences that can potentially be generated. This high diversity allows T cells to recognize a large number of epitopes displayed on different MHC alleles.

### SUMMARY

Provided are agents and methods for preventing or inhibiting a multiple sclerosis (MS)-associated adaptive immune response in a subject in need thereof. In some instances, the agents comprise or consist of a soluble peptide-major histocompatibility complex (pMHC) or multimer(s) thereof. The peptide of the pMHC is one identified as being targeted by MS-associated T-cell receptors (TCRs). The agents may be used alone or may be conjugated or fused to one or more moieties that result in the targeted depletion of T cells expressing the MS-associated TCRs. Other agents such as the MS-associated TCRs or binding domains thereof, and inhibitory immune cells expressing such TCRs are also provided. Aspects of the present disclosure further include methods of treating multiple sclerosis in a subject in need thereof via administration of an agent of the present disclosure.

### BRIEF DESCRIPTION OF THE FIGURES

FIG. 1: Normalized median CD69 expression levels by TCR1-expressing cells co-stimulated with peptide containing a fixed amino acid at each position

FIG. 2: TCR-of-interest-expressing cells were co-stimulated with HLA-DRB1*15:01-expressing K562s loaded with peptide at the concentrations of 50⁴, 10⁴, 10³, 10², 10¹, 10⁰, 10⁻¹, 10⁻², 10⁻³, 10⁻⁴, 10⁻⁵ nM. Dose response of TCRs to the peptide, MEETAIWEQHTVTLHRA (SEQ ID NO: 6). TCR1 is MS_DR15_ESg1_2 (an MS-associated TCR). TCR2 is MS_DR15_ESg1_Mun1 (an MS-associated TCR). TCR3 is MS_DR15_ESg1_Mun1e1 (an MS-associated TCR and negative control).

FIG. 3: Data from an experiment in which T cells expressing various TCRs were co-cultured with HLA-DRB1*15:01 positive cells expressing various antigen-encoding constructs. Antigen encoding constructs (1)-(5) were as follows: (1) Mock; (2) ZO-1; (3) ZO-1_MBP; (4) ZO-1_COVID; and (5) MBP. For each condition (antigen encoding constructs (1)-(5)), the bars from left to right indicate T cell activation via CD137 expression from the following TCRs: (a) mock; (b) MS_DR15_ESg1_Mun1 (an MS-associated TCR); (c) MS_DR15_ESg1_2 (an MS-associated TCR); (d) MS_DR15_ESg1_1e4 (an MS-associated TCR); (e) MS_DR15_ESg1_Mun1e1 (an MS-associated TCR); Ob. 1A12 (MBP-specific); and NL228_COVID_1 (COVID-specific).

FIG. 4A-4C: Deorphanization data for peptides PXDC1_YGL15 and HuTV_TPY15 for ESg1_1, ESg1_2 and ESg1_Mun1, respectively.

FIG. 5A-5C: Deorphanization data for ZO-2 and ZO-3 peptides for ESg1_1, ESg1_2 and ESg1_Mun1, respectively.

FIG. 6: Deorphanization data for peptides Synthetic (Syn-2) (indicated by *), POLN_IPI-15 (indicated by **), ZO-1 (indicated by ***) and no peptide control (DMSO) for ESg1_1, ESg1_2 and ESg1_Mun1 TCRs.

### DETAILED DESCRIPTION

Before the agents and methods of the present disclosure are described in greater detail, it is to be understood that the agents and methods are not limited to particular embodiments described, as such may, of course, vary. It is also to be understood that the terminology used herein is for the purpose of describing particular embodiments only, and is not intended to be limiting, since the scope of the agents and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the agents and methods. The upper and lower limits of these smaller ranges may independently be included in the smaller ranges and are also encompassed within the agents and methods, subject to any specifically excluded limit in the stated range. Where the stated range includes one or both of the limits, ranges excluding either or both of those included limits are also included in the agents and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the agents and methods belong. Although any agents and methods similar or equivalent to those described herein can also be used in the practice or testing of the agents and methods, representative illustrative agents and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present agents and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the agents and methods, which are, for clarity, described in the context of separate embodiments, may also be provided in combination in a single embodiment. Conversely, various features of the agents and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present agents and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Agents for Preventing or Inhibiting Ms-Associated Adaptive Immune Responses

Aspects of the present disclosure include agents for preventing or inhibiting multiple sclerosis (MS)-associated adaptive immune responses. The agents and therapeutic uses thereof are based in part on the discovery of MS-associated (e.g., MS-specific) TCRs sharing the TCRB motif CAISESW [T/A]GG[T/S]DTQYF, and in turn the discovery of antigens to which those TCRs bind with HLA-DRB1*15. Thus, with the benefit of the present disclosure, it will be appreciated that a variety of agents and therapeutic approaches for preventing or inhibiting MS-associated adaptive immune responses (e.g., in HLA-DRB1*15 positive subjects) are provided based on the structural information of the discovered MS-associated TCRs and peptide-HLA complexes to which they bind. Non-limiting examples include peptide-major histocompatibility complexes (pMHCs) or multimers thereof which may be administered to a subject having MS, where the pMHCs or multimers thereof compete for binding to the MS-associated TCRs with the endogenous complex bound by those TCRs in the absence of the pMHCs or multimers thereof. Such pMHCs may be conjugated or fused to an agent that binds to T cells expressing the MS-associated TCRs and inhibits activation and/or depletes such T cells. Further non-limiting examples include agents that induce tolerance to the antigen bound by the MS-associated TCRs, e.g., regulatory immune cells (e.g., Tregs) expressing the MS-associated TCR, tolerizing vaccines, agents that reprogram the MS-associated T cells to differentiate and expand into inhibitory immune cells (e.g., agents such as Navicims and the like), agents such as antibodies that target the MS-associated TCRs (e.g., via their shared TCRBV10 region) as well as PROteolysis TArgeting Chimeras (PROTACs), immune modulating monoclonal TCR against autoimmunity (ImmTAAI) agents, and many others. Also provided are gene silencing and gene editing compositions for inhibiting or eliminating expression of the MS-associated TCRs. Details regarding embodiments of the agents and compositions of the present disclosure will now be provided.

pMHC-Based Agents

Aspects of the present disclosure include soluble peptide-major histocompatibility complexes (pMHCs) or multimers thereof. The soluble pMHCs or multimers thereof do not occur in nature. For example, by “soluble” in this context is meant the pMHC or multimer thereof is not present on the surface of an antigen-presenting cell (APC). In some embodiments, the MHC is HLA-DRB1*15 and the peptide comprises, consists essentially of, or consists of an amino acid sequence chosen from TAIWEQHTV (SEQ ID NO: 7), IALWESHDV (SEQ ID NO:9), VAIKEAHDI (SEQ ID NO: 11), IGLAESHDN (SEQ ID NO: 13), ELIWEQYTV (SEQ ID NO: 15), LTIWEQHTA (SEQ ID NO: 17), and LAVMESHAI (SEQ ID NO: 19). By “consists essentially of” is meant the peptide has one or two additional N- and/or C-terminal amino acids, or one or two fewer N- and/or C-terminal amino acids, as compared to the reference sequence.

The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

The term “amino acid” includes, but is not limited to, naturally-occurring amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid). A peptide or pMHC of the present disclosure may comprise only of L-amino acids, may comprise only of D-amino acids, or may comprise a mixture of L-amino acids and D-amino acids. Peptides or pMHCs comprised of D-amino acids (e.g., all-D amino acids) may be advantageous compared to their all-L counterparts in terms of PK, reduced immunogenicity, and/or the like. See Mandal et al. (2012) PNAS 109:14779-14784. Moreover, any of the peptides or pMHCs of the present disclosure may comprise one or more beta amino acids, one or more gamma amino acids, and/or one or more of any other amino acid types that suitably replace alpha-amino acids. See, e.g., Sang et al. Acc. Chem. Res. 53, 10, 2425-2442. In certain embodiments, a peptides or pMHC of the present disclosure comprises one or more amino acid analogs available from Bachem.

In some instances, the peptide of the pMHC or multimer thereof has a length in a range of from 5-30 amino acids (e.g., 5-25, 5-20, 5-18, 5-17, 5-15, 7-30, 7-25, 7-20, 7-18, 7-17, 7-15, 8-30, 8-25, 8-20, 8-18, 8-17, 8-15, 9-30, 9-25, 9-20, 9-18, 9-17, 9-15, 10-30, 10-25, 10-20, 10-18, 10-17, or 10-15 amino acids). In some cases, the peptide has a length in a range of from 7-20 amino acids (e.g., 7-11, 8-10, 15-19, 16-18, or 9-17 amino acids). In some cases, the peptide has a length in a range of from 8-10 amino acids. In some cases, the peptide has a length in a range of from 16-18 amino acids. In some cases, the peptide has a length of 8, 9, 10, 11 or 12 amino acids, e.g., 9 amino acids.

In certain embodiments, the peptide of the pMHC or multimer thereof comprises, consists essentially of, or consists of the amino acid sequence TAIWEQHTV (SEQ ID NO: 7). According to some embodiments, the peptide of the pMHC or multimer thereof comprises, consists essentially of, or consists of the amino acid sequence IALWESHDV (SEQ ID NO:9). In some instances, the peptide of the pMHC or multimer thereof comprises, consists essentially of, or consists of the amino acid sequence VAIKEAHDI (SEQ ID NO: 11). In certain embodiments, the peptide of the pMHC or multimer thereof comprises, consists essentially of, or consists of the amino acid sequence IGLAESHDN (SEQ ID NO: 13). According to some embodiments, the peptide of the pMHC or multimer thereof comprises, consists essentially of, or consists of the amino acid sequence ELIWEQYTV (SEQ ID NO: 15). In some instances, the peptide of the pMHC or multimer thereof comprises, consists essentially of, or consists of the amino acid sequence LTIWEQHTA (SEQ ID NO: 17). In certain embodiments, the peptide of the pMHC or multimer thereof comprises, consists essentially of, or consists of the amino acid sequence LAVMESHAI (SEQ ID NO: 19).

In some instances, any of the aforementioned peptides may comprise one or two amino acid substitutions. Examples of such substitutions include conservative and/or non-conservative amino acid substitutions which may be incorporated into a peptide followed by screening for a desired property, e.g., retained/improved binding to the MS-associated TCR(s) (e.g., when complexed with HLA-DRB1*15), and/or any other properties of interest. Conservative substitutions are shown in Table 1. More substantial changes are provided in Table 1 under the heading of “exemplary substitutions,” and as further described below in reference to amino acid side chain classes.

TABLE 1
Amino Acid Substitutions

| Original Residue | Exemplary Substitutions | Conservative Substitutions |
|---|---|---|
| Ala (A) | Val; Leu; Ile | Val |
| Arg (R) | Lys; Gln; Asn | Lys |
| Original Residue | Exemplary Substitutions | Preferred Substitutions |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

A peptide of the pMHCs or multimers thereof may be produced using any convenient approach. Where a peptide is chemically synthesized, the synthesis may proceed via liquid-phase or solid-phase. Solid-phase synthesis (SPPS) allows the incorporation of unnatural amino acids, peptide/protein backbone modification. Various forms of SPPS, such as Fmoc and Boc, are available for synthesizing the peptides of the present disclosure. Details of the chemical synthesis are known in the art (e.g., Ganesan A. 2006 Mini Rev. Med Chem. 6:3-10 and Camarero J A et al. 2005 Protein Pept Lett. 12:723-8). Briefly, small insoluble, porous beads are treated with functional units on which peptide chains are built. After repeated cycling of coupling/deprotection, the free N-terminal amine of a solid-phase attached peptide or amino acid is coupled to a single N-protected amino acid unit. This unit is then deprotected, revealing a new N-terminal amine to which a further amino acid may be attached. The peptide remains immobilized on the solid-phase and may undergo a filtration process before being cleaved off.

Peptides or polypeptides (e.g., HLA complex subunits or single chain versions thereof) of the present disclosure may also be produced by recombinant methods. Where the peptide or polypeptide is produced using recombinant techniques, a peptide or polypeptide may be produced as an intracellular protein or as a secreted protein, using any suitable construct and any suitable host cell, which can be a prokaryotic or eukaryotic cell, such as a bacterial (e.g., E. coli, including but not limited to E. coli strains engineered for incorporation of non-natural amino acids) or a yeast host cell, respectively. Other examples of eukaryotic cells that may be used as host cells include insect cells, mammalian cells, and/or plant cells. Where mammalian host cells are used, the cells may include one or more of the following: human cells (e.g. HeLa, 293, H9, and Jurkat cells); mouse cells (e.g., X3, NIH3T3, pancreatic ductal adenocarcinoma 2.1, L cells, and C127 cells); primate cells (e.g. Cos 1, Cos 7, and CV1) and hamster cells (e.g., Chinese hamster ovary (CHO) cells).

A wide range of host-vector systems suitable for the expression of the peptide or polypeptide may be employed according to standard procedures known in the art. See, e.g., Sambrook et al. 1989 Current Protocols in Molecular Biology Cold Spring Harbor Press, New York and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced peptide- or polypeptide-encoding nucleic acid. The peptide- or polypeptide-encoding nucleic acid can be provided as an inheritable episomal element (e.g., a plasmid) or can be genomically integrated. A variety of appropriate vectors for use in production of a polypeptide of interest are available commercially.

Vectors can provide for extrachromosomal maintenance in a host cell or can provide for integration into the host cell genome. The expression vector provides transcriptional and translational regulatory sequences, and may provide for inducible or constitutive expression, where the coding region is operably linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like). Expression constructs generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition the expression construct may contain a selectable marker gene to allow the selection of transformed host cells. Selectable genes are well known in the art and will vary with the host cell used.

Isolation and purification of the peptide or polypeptide can be accomplished according to methods known in the art. For example, a peptide or polypeptide can be isolated from a lysate of cells genetically modified to express the protein constitutively and/or upon induction, or from a synthetic reaction mixture, by immunoaffinity purification, which generally involves contacting the sample with an anti-peptide or anti-polypeptide antibody (or in the case of a tagged polypeptide, an anti-tag antibody), washing to remove non-specifically bound material, and eluting the specifically bound polypeptide. The isolated peptide or polypeptide can be further purified by dialysis and other methods normally employed in protein purification methods. In one embodiment, the peptide or polypeptide may be isolated using metal chelate chromatography methods. Peptides or polypeptides of the present disclosure may contain modifications (e.g., affinity tags or the like) to facilitate isolation.

The peptides or polypeptides may be prepared in substantially pure or isolated form (e.g., free from other polypeptides). The peptides or polypeptides (or complexes thereof) can be present in a composition that is enriched for the polypeptide relative to other components that may be present (e.g., other polypeptides or other host cell components). Purified peptide or polypeptide may be provided such that the peptide or polypeptide (or complex thereof) is present in a composition that is substantially free of other expressed proteins, e.g., less than 98%, less than 95%, less than 90%, less than 80%, less than 60%, or less than 50%, of the composition is made up of other expressed proteins.

Because of the knowledge of the codons corresponding to the various amino acids, availability of an amino acid sequence of a peptide or polypeptide of interest provides a description of all the polynucleotides capable of encoding the peptide or polypeptide of interest. The degeneracy of the genetic code, where the same amino acids are encoded by alternative or synonymous codons allows an extremely large number of nucleic acids to be made, all of which encode the peptide or polypeptide and domains disclosed herein. Thus, having identified a particular amino acid sequence, those of ordinary skill in the art could make any number of different nucleic acids by simply modifying the sequence of one or more codons in a way which does not change the amino acid sequence of the peptide or polypeptide of interest. In this regard, the present disclosure specifically contemplates each and every possible variation of polynucleotides that could be made by selecting combinations based upon the possible codon choices, and all such variations are to be considered specifically disclosed for any peptide or polypeptide disclosed herein.

Approaches for loading peptides into MHCs to produce pMHC complexes are known and include the Flex-T™ loading method (BioLegend) which involves loading of peptides of interest into the binding site of the MHC groove, by using ultraviolet (UV) light labile, exchangeable peptides. MHC monomers may be loaded with a peptide that can be degraded by the use of a UV light source. This allows for a peptide exchange when the UV irradiation is done in the presence of the peptide of interest (which is not UV-labile). pMHC monomers comprising the desired MHC-peptide complex may also be ordered and obtained from vendors such as Biolegend®, ProImmune, and the like.

In some embodiments, a peptide and MHC of a pMHC complex are covalently linked. In one non-limiting example, the peptide and MHC molecules are provided in a single polypeptide chain in which the peptide and MHC are fused directly or indirectly, e.g., via one or more linkers. By “flexible” is meant the linkers are of sufficient length and flexibility to provide flexibility between the peptide and MHC and permit loading of the peptide into the binding site of the MHC groove. Flexible linkers are often rich in small or polar amino acids such as Gly and Ser to provide good flexibility and solubility. In certain embodiments, a Gly-Ser (GS) linker is employed, non-limiting examples of which are those comprising one or more GGGGS units, i.e., (GGGGS), where n is an integer of 1 or greater, e.g., 1 to 10. In some embodiments, a flexible linker comprising Gly and Ser, and one or more threonine (Thr) residues is employed. According to some embodiments, a flexible linker comprising Gly or Ser residues, and one or more Thr residues is employed.

Any of the pMHC complexes of the present disclosure may be provided in multimeric form. As used herein, a “multimer” is two or more of the same or different type of pMHC of interest or stably associated with one another. By “stably associated” is meant a physical association between two entities in which the mean half-life of association is one day or more in PBS at 4° C. In certain embodiments, the physical association between the two entities has a mean half-life of one day or more, one week or more, one month or more, including six months or more, e.g., 1 year or more, in PBS at 4° C. According to some embodiments, the stable association arises from a covalent bond between the two entities, a non-covalent bond between the two entities (e.g., an ionic or metallic bond), or other forms of chemical attraction, such as hydrogen bonding, Van der Waals forces, and/or the like.

The pMHC multimers of the present disclosure may include any desired number of pMHC monomers. In certain embodiments, the multimers comprise n monomers, wherein n is an integer from 2 to 100, 2 to 75, 2 to 50, 2 to 40, 2 to 30, 2 to 20, 2 to 15, or 2 to 10, e.g., 10, 9, 8, 7, 6 (hexamers), 5 (pentamers), 4 (tetramers), 3 (trimers) or 2 (dimers). According to some embodiments, the multimers are tetramers. In certain embodiments, the multimers are pentamers.

A variety of approaches are available to multimerize the pMHCs of the present disclosure. In certain embodiments, the multimers are stably associated via non-covalent interactions. For example, the monomers may be biotin-labeled, and multimerization is achieved using streptavidin or a streptavidin-labeled molecule to which multiple biotin-labeled monomers stably associate. In one non-limiting example, a dextran-backbone conjugated with streptavidin (e.g., Streptavidin-Dextramer® (Immudex)) is employed to multimerize two or more (e.g., 4 or more, 5 or more, 10 or more, etc.) biotin-labeled pMHC monomers. Details regarding classes and types of pMHC multimers and methods of making such multimers are described, e.g., in Chang (2021) MHC Multimer: A Molecular Toolbox for Immunologists Mol Cells. 44 (5): 328-334, the disclosure of which is incorporated herein by reference in its entirety for all purposes.

In some embodiments, a pMHC or multimer thereof is conjugated or fused to a T cell inhibitor. The term “conjugated” generally refers to a chemical linkage, either covalent or non-covalent, usually covalent, that proximally associates one molecule of interest with a second molecule of interest. The site of conjugation or fusion generally should not overlap with the combined region of the peptide and MHC bound by the MS-associated TCRs so as to not interfere with binding of the TCRs to the pMHC or multimer thereof.

In some embodiments, the T cell inhibitor is an agonist of an immune checkpoint molecule. Non-limiting examples of such agonists include agonists of programmed cell death-1 (PD-1), cytotoxic T-lymphocyte-associated antigen 4 (CTLA-4), T cell immunoreceptor with Ig and ITIM domains (TIGIT), T-cell immunoglobulin domain and mucin domain 3 (TIM-3), V-domain Ig suppressor of T cell activation (VISTA), or B and T lymphocyte attenuator (BTLA). In some instances, the agonist is a ligand for the immune checkpoint molecule. For example, the agonist may be PD-L1 when the immune checkpoint molecule is PD-1.

According to certain embodiments, the T cell inhibitor is an anti-CD3 binding agent, e.g., an anti-CD3 antibody.

In some instances, the T cell inhibitor or any other agent conjugated or fused to the pMHC or multimer thereof is an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the target molecule (e.g., a cell surface molecule of a target cell), including, but not limited to single chain Fv (scFv), Fab, (Fab′) 2, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, single chain Fv (scFv), Fab, (Fab)₂, (scFv′)₂, or a single variable domain located on a heavy chain (VHH). According to some embodiments, the antibody is a VHH (sometimes referred to as a “nanobody”).

In certain embodiments, the agent conjugated to the pMHC or multimer thereof is conjugated to a chemotherapeutic agent, a toxin, a radiation-sensitizing agent, a radioactive isotope (e.g., a therapeutic radioactive isotope), a detectable label, and a half-life extending moiety. According to some embodiments, the agent is a therapeutic agent, e.g., a chemotherapeutic agent. Therapeutic agents of interest include agents capable of affecting the function of T cells (e.g., CD4 T cells) that bind the pMHC or multimer thereof. When the function of the cell/tissue is pathological, an agent that reduces the function of the cell/tissue may be employed. In certain embodiments, a conjugate of the present disclosure includes an agent that reduces the function of a T cell that binds to the pMHC or multimer thereof by inhibiting cell proliferation and/or killing the cell. Such agents may vary and include cytostatic agents and cytotoxic agents, e.g., an agent capable of killing a target cell tissue with or without being internalized into a target cell.

In certain embodiments, the therapeutic agent is a cytotoxic agent selected from an enediyne, a lexitropsin, a duocarmycin, a taxane, a puromycin, a dolastatin, a maytansinoid, and a vinca alkaloid. In some embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to some embodiments, the agent is a toxin, such as a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the agent is a radiation-sensitizing agent. As used herein, a “radiation-sensitizing agent” is an agent that enhances the ability of radiation to kill T cells. Non-limiting examples of radiation-sensitizing agents that may be conjugated to the pMHC or multimer thereof include cisplatin, 5-fluorouracil (5-FU), AZD7762, selumetinib, and the like. In certain embodiments, the agent is a radioisotope, e.g., useful for therapy and/or detection (e.g., imaging). Non-limiting examples of radioisotopes that may be conjugated to the pMHC or multimer thereof include but are not limited to ²²⁵Ac, ¹¹¹Ag, ¹¹⁴Ag, ⁷¹As, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ¹³C, ⁵⁵Co, ⁶²Cu, ⁶⁴Cu, ⁶⁷Cu, ¹⁶⁵Dy, ¹⁶⁶Dy, ¹⁶⁹Er, ¹⁸F, ¹⁹F, ⁵²Fe, ⁵⁹Fe, ⁶⁶Ga, ⁶⁷Ga, ⁶⁸Ga, ⁷²Ga, ¹⁵⁴⁻¹⁵⁸Gd, ¹⁵⁷Gd, ¹⁵⁹Gd, ¹⁶⁶Ho, ¹²⁰I, ¹²¹I, ¹²³I, ¹²⁴I, ¹²⁵I, ¹³¹I, ¹¹⁰In, ¹¹¹In, ^{113m}In, ¹⁹⁴Ir, ^{81m}Kr, ¹⁷⁷Lu, ⁵¹Mn, ⁵²Mn, ⁹⁹Mo, ¹³N, ¹⁵N, ¹⁵O, ¹⁷O, ³²P, ³³P, ²¹¹Pb, ²¹²Pb, ¹⁰⁹Pd, ¹⁴⁹Pm, ¹⁵¹Pm, ¹⁴²Pr, ¹⁴³Pr, ¹⁹¹PT, ^{193m}PT, ^{195m}Pt, ²²³Ra, ¹⁴²Rb, ¹⁸⁶Re, ¹⁸⁸Re, ¹⁸⁹Re, ¹⁰⁵Rh, ⁴⁷Sc, ⁷⁵Se, ¹⁵³Sm, ^{117m}Sn, ¹²¹Sn, ⁸³Sr, ⁸⁹Sr, ¹⁶¹Tb, ⁹⁴Tc, ⁹⁹Tc, ^{99m}Tc, ²²⁷Th, ²⁰¹Tl, ¹⁷²Tm, ¹²⁷Te, ⁹⁰Y, ¹⁶⁹Yb, ¹⁷⁵Yb, ¹³³X, and ⁸⁹Zr.

In certain embodiments, a radioisotope is conjugated to the pMHC or multimer thereof via a chelator, for example, a bifunctional chelator. A bifunctional chelator may contain a metal chelating moiety that binds the radioisotope in a stable coordination complex and a reactive functional group that is covalently linked to a pMHC or multimer thereof, so that the radioisotope may be properly directed to the desirable T cells in vivo. Non-limiting examples of bifunctional chelators that may be employed to conjugate a pMHC or multimer thereof of the present disclosure to a radioisotope include p-SCN-Bn-DOTA and p-SCN-Bn-deferoxamine. Additional examples of bifunctional chelators that may be employed to conjugate a pMHC or multimer thereof of the present disclosure to a radioisotope include those described in Price & Orvig (2014) Chem. Soc. Rev. 43:260; and Brechbiel (2008) Q J Nucl Med Mol Imaging 52 (2): 166-173.

According to some embodiments, the radioisotope is a therapeutic radioisotope. In certain embodiments, the radioisotope is an alpha emitting radioisotope, e.g., ²²⁵Ac, ²¹¹At, ²¹²Bi/²¹²Pb, ²¹³Bi, ²²³Ra, or ²²⁷Th. In other embodiments, the radioisotope is a beta minus emitting radioisotope, e.g., ³²P, ³³P, ⁶⁷Cu, ⁹⁰Y, ¹³¹I or ¹⁷⁷Lu.

According to some embodiments, the agent is a labeling agent. By “labeling agent” (or “detectable label”) is meant the agent detectably labels the pMHC or multimer thereof, such that the pMHC or multimer thereof may be detected in an application of interest (e.g., clinical applications). Detectable labels of interest include radioisotopes (e.g., gamma or positron emitters), enzymes that generate a detectable product (e.g., horseradish peroxidase, alkaline phosphatase, luciferase, etc.), fluorescent proteins, paramagnetic atoms, and the like. In certain embodiments, the pMHC or multimer thereof is conjugated to a specific binding partner of detectable label, e.g., conjugated to biotin such that detection may occur via a detectable label that includes avidin/streptavidin.

In certain embodiments, the agent is a labeling agent that finds use in in vivo imaging, such as near-infrared (NIR) optical imaging, single-photon emission computed tomography (SPECT)+CT imaging, positron emission tomography (PET)+CT imaging, nuclear magnetic resonance (NMR) spectroscopy, or the like. Labeling agents that find use in such applications include, but are not limited to, fluorescent labels, radioisotopes, and the like. In certain aspects, the labeling agent is a multi-modal in vivo imaging agent that permits in vivo imaging using two or more imaging approaches (e.g., see Thorp-Greenwood and Coogan (2011) Dalton Trans. 40:6129-6143).

In certain embodiments, the labeling agent is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 800CW Fluors. According to some embodiments, the labeling agent is an in vivo imaging agent that finds use in SPECT imaging applications, non-limiting examples of which include ^{99m}Tc, ¹¹¹In, ¹²³I, ²⁰¹Tl, and ¹³³Xe. In certain embodiments, the labeling agent is an in vivo imaging agent that finds use in PET imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

For half-life extension, the pMHCs or multimers thereof of the present disclosure may be conjugated to an agent that provides for an improved pharmacokinetic profile (e.g., by PEGylation, hyperglycosylation, and the like). Modifications that can enhance serum half-life are of interest. A subject pMHC or multimer thereof may be “PEGylated”, as containing one or more poly(ethylene glycol) (PEG) moieties. Methods and reagents suitable for PEGylation of a protein are well known in the art and may be found, e.g., in U.S. Pat. No. 5,849,860. PEG suitable for conjugation to a protein is generally soluble in water at room temperature and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons. The PEG conjugated to the subject pMHC or multimer thereof can be linear. The PEG conjugated to the subject pMHC or multimer thereof may also be branched. Branched PEG derivatives such as those described in U.S. Pat. No. 5,643,575, “star-PEGs” and multi-armed PEGs. Star PEGs are described in the art including, e.g., in U.S. Pat. No. 6,046,305.

Aspects of the present disclosure also include agents that comprise one or more of the pMHCs or multimers thereof of the present disclosure. In one non-limiting example, provided are nanoparticles to which one or more of the pMHCs or multimers thereof are linked, e.g., conjugated. Nanoparticles comprising pMHCs or multimers thereof and approaches for making and using the same in therapy are known and described, e.g., in Singha et al. (2017) Nature Nanotechnology 12:701-710, the disclosure of which is incorporated herein by reference in its entirety for all purposes. Available chemistries for linking pMHCs or multimers thereof to nanoparticles include click chemistry, EDC/NHS chemistry, maleimide-cysteine chemistry, OPSS-cysteine chemistry, and the like.

### Inhibitory Immune Cells Expressing MS-Associated TCRs

Aspects of the present disclosure further include genetically modified cells that express a T cell receptor (TCR) comprising a TCRB chain comprising a TCRβ CDR3 sequence of one of SEQ ID NOs: 1-3. In some instances, the TCRβ CDR3 sequence is CAISESWTGGSDTQYF (SEQ ID NO: 1) and the TCRα CDR3 sequence is CIVRPNTGTASKLTF (SEQ ID NO: 4). In other embodiments, the TCRβ CDR3 sequence is CAISESWAGGTDTQYF (SEQ ID NO: 2) and the TCRα CDR3 sequence is CIVRGNTGTASKLTF (SEQ ID NO: 5). In yet other embodiments, the TCRβ CDR3 sequence is CAISEGWTGNTDTQYF (SEQ ID NO: 3) and the TCRα CDR3 sequence is CIVRGNTGTASKLTF (SEQ ID NO: 5).

In certain embodiments, the cell is an immune regulatory cell. Non-limiting examples of immune regulatory cells of the present disclosure include regulatory T cells (Tregs) and regulatory B cells (Bregs). Approaches for introducing constructs of interest into cells of interest are known in the art and described elsewhere herein.

As will be appreciated with the benefit of the present disclosure, immune regulatory cells such a Tregs expressing the MS-associated TCRs described herein find use in promoting immune tolerance, e.g., suppressing the functions of T cells expressing the MS-associated TCRs.

### TCR Expression

In some embodiments, the expression of an MS-associated TCR in T cells is reduced/inhibited or eliminated by, e.g., genome editing, gene deletion, gene disruption, miRNA, siRNA, shRNA, antisense oligonucleotides, or by other means of knocking down TCR expression. See, e.g., Zhang and Morgan, Adv Drug Deliv Rev. (2012) 64 (8): 756-762. As such, in some embodiments, a subject agent is one that inhibits expression of an MS-associated TCR.

Expression of an MS-associated TCR (e.g., a TCRA and/or TCRβ subunit) may be inhibited or blocked by any convenient approach, e.g., gene deletion, gene disruption, genome editing, miRNA, siRNA, shRNA, antisense, and the like. In some embodiments, a subject agent genetically modifies a gene encoding an MS-associated TCR (e.g., TCRA and/or TCRB), where the genetic modification prevents expression of the MS-associated TCR and/or mutates the gene such that the MS-associated TCR no longer binds its pMHC target. Gene modification (gene deletion, gene disruption, genome editing) may be achieved using a programmable genome editing nuclease, such as a zinc finger nuclease (ZFN), TALE-nuclease (TALEN), or CRISPR/Cas nuclease. For example, zinc fingered proteins (ZFs) or TALEs may be fused to nuclease domains to generate ZFNs and TALENs, which recognize their intended nucleic acid target through their engineered binding domains and cleave DNA via the nuclease activity. With regard to CRISPR/Cas proteins, in class 2 CRISPR systems, the functions of the effector complex (e.g., the cleavage of target DNA) are carried out by a single protein (which can be referred to as a CRISPR/Cas effector protein)—where the natural protein is an endonuclease (e.g., see Zetsche et al, Cell. 2015 Oct. 22; 163 (3): 759-71; Makarova et al, Nat Rev Microbiol. 2015 November; 13 (11): 722-36; Shmakov et al., Mol Cell. 2015 Nov. 5; 60 (3): 385-97; Shmakov et al., Nat Rev Microbiol. 2017 March; 15 (3): 169-182: “Diversity and evolution of class 2 CRISPR-Cas systems”; and Koonin et al., Curr Opin Microbiol. 2017 June: 37:67-78). As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c, Cas12d/CasY, Cas12e/CasX), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

A nucleic acid that binds to a class 2 CRISPR/Cas effector protein (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to as a targeting sequence), which is a nucleotide sequence that is complementary to a sequence of a target nucleic acid. A guide RNA can be referred to by the protein to which it corresponds. For example, when the class 2 CRISPR/Cas effector protein is a Cas9 protein, the corresponding guide RNA can be referred to as a “Cas9 guide RNA.” Likewise, as another example, when the class 2 CRISPR/Cas effector protein is a Cpf1 protein, the corresponding guide RNA can be referred to as a “Cpf1 guide RNA.”

As will be known to one of ordinary skill in the art, in some embodiments, a guide RNA includes two separate nucleic acid molecules: an “activator” and a “targeter” and is referred to as a “dual guide RNA”, a “double-molecule guide RNA”, a “two-molecule guide RNA”, or a “dgRNA.”

In some embodiments, the guide RNA is one molecule (e.g., for some class 2 CRISPR/Cas proteins, the corresponding natural guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some embodiments, a programmable genome editing protein such as a CRISPR/Cas effector protein, a ZF, or TALE is fused to a heterologous protein having any desired activity such as DNA-modifying activity (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity); transcription modulation activity (e.g., fusion to a transcription repressor or transcription activator); an activity that modifies a protein (e.g., a histone) that is associated with target DNA (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some such cases the CRISPR/Cas effector protein harbors a mutation that reduces the endogenous nuclease activity (e.g., in some cases renders it a nickase and in some cases renders it catalytically inactive (“dead), e.g., a dCas9). In some cases, a CRISPR/Cas effector protein (e.g., a nickase or ‘dead’ version) is fused to a heterologous protein that has transcription repressor activity (e.g., includes a transcription repression domain) and thereby reduces transcription (and therefore expression) of a target gene (e.g., one encoding an MS-associated TCR).

Examples of proteins (or fragments thereof) that can be used as heterologous proteins to decrease transcription include but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like

The CRISPR/Cas system can thus be used to edit a TCR (e.g., adding or deleting a basepair), or introducing a premature stop which thus decreases expression of a TCR. The CRISPR/Cas system can alternatively be used like RNA interference, turning off TCR gene expression (e.g., in a reversible fashion).

For additional information related to programmable gene editing tools (e.g., CRISPR/Cas RNA-guided proteins such as Cas9, CasX, CasY, and Cpf1, Zinc finger proteins such as Zinc finger nucleases, TALE proteins such as TALENs, CRISPR/Cas guide RNAs, PAMs, and the like) refer to, for example, Dreier, et al., (2001) J Biol Chem 276:29466-78; Dreier, et al., (2000) J Mol Biol 303:489-502; Liu, et al., (2002) J Biol Chem 277:3850-6); Dreier, et al., (2005) J Biol Chem 280:35588-97; Jamieson, et al., (2003) Nature Rev Drug Discov 2:361-8; Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29:183-212; Segal and Barbas, (2001) Curr Opin Biotechnol 12:632-7; Segal, et al., (2003) Biochemistry 42:2137-48; Beerli and Barbas, (2002) Nat Biotechnol 20:135-41; Carroll, et al., (2006) Nature Protocols 1:1329; Ordiz, et al., (2002) Proc Natl Acad Sci USA 99:13290-5; Guan, et al., (2002) Proc Natl Acad Sci USA 99:13296-301; Sanjana et al., Nature Protocols, 7:171-192 (2012); Zetsche et al, Cell. 2015 Oct. 22; 163 (3): 759-71; Makarova et al, Nat Rev Microbiol. 2015 November; 13 (11): 722-36; Shmakov et al., Mol Cell. 2015 Nov. 5; 60 (3): 385-97; Jinek et al., Science. 2012 Aug. 17; 337 (6096): 816-21; Chylinski et al., RNA Biol. 2013 May; 10 (5): 726-37; Ma et al., Biomed Res Int. 2013; 2013:270805; Hou et al., Proc Natl Acad Sci USA. 2013 September 24; 110 (39): 15644-9; Jinek et al., Elife. 2013; 2: e00471; Pattanayak et al., Nat Biotechnol. 2013 September; 31 (9): 839-43; Qi et al, Cell. 2013 Feb. 28; 152 (5): 1173-83; Wang et al., Cell. 2013 May 9; 153 (4): 910-8; Auer et. al., Genome Res. 2013 Oct. 31; Chen et. al., Nucleic Acids Res. 2013 Nov. 1; 41 (20): e19; Cheng et. al., Cell Res. 2013 October; 23 (10): 1163-71; Cho et. al., Genetics. 2013 November; 195 (3): 1177-80; DiCarlo et al., Nucleic Acids Res. 2013 April; 41 (7): 4336-43; Dickinson et. al., Nat Methods. 2013 October; 10 (10): 1028-34; Ebina et. al., Sci Rep. 2013; 3:2510; Fujii et. al, Nucleic Acids Res. 2013 Nov. 1; 41 (20): e187; Hu et. al., Cell Res. 2013 November; 23 (11): 1322-5; Jiang et. al., Nucleic Acids Res. 2013 Nov. 1; 41 (20): e188; Larson et. al., Nat Protoc. 2013 November; 8 (11): 2180-96; Mali et. at., Nat Methods. 2013 October; 10 (10): 957-63; Nakayama et. al., Genesis. 2013 December; 51 (12): 835-43; Ran et. al., Nat Protoc. 2013 November; 8 (11): 2281-308; Ran et. al., Cell. 2013 Sep. 12; 154 (6): 1380-9; Upadhyay et. al., G3 (Bethesda). 2013 Dec. 9; 3 (12): 2233-8; Walsh et. al., Proc Natl Acad Sci USA. 2013 Sep. 24; 110 (39): 15514-5; Xie et. al., Mol Plant. 2013 Oct. 9; Yang et. al., Cell. 2013 Sep. 12; 154 (6): 1370-9; Briner et al., Mol Cell. 2014 Oct. 23; 56 (2): 333-9; Burstein et al., Nature. 2016 Dec. 22-Epub ahead of print; Gao et al., Nat Biotechnol. 2016 July 34 (7): 768-73; Shmakov et al., Nat Rev Microbiol. 2017 March; 15 (3): 169-182; as well as international patent application publication Nos. WO2002099084; WO00/42219; WO02/42459; WO2003062455; WO03/080809; WO05/014791; WO05/084190; WO08/021207; WO09/042186; WO09/054985; and WO10/065123; U.S. patent application publication Nos. 20030059767, 20030108880, 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; 20140377868; 20150166983; and 20160208243; and U.S. Pat. Nos. 6,140,466; 6,511,808; 6,453,242 8,685,737; 8,906,616; 8,895,308; 8,889,418; 8,889,356; 8,871,445; 8,865,406; 8,795,965; 8,771,945; and 8,697,359; all of which are hereby incorporated by reference in their entirety.

In some cases, a gene editing composition includes a CRISPR/Cas base editor (e.g. Komor et al (2016) Nature. 533 (7603): 420-424. doi: 10.1038/nature17946). In some cases, a gene editing composition includes a CRISPR/Cas prime editor (e.g., Anzalone et al (2019) Nature 576:149-157 https://doi.org/10.1038/s41586-019-1711-4).

In some embodiments, TCR expression is inhibited using an RNAi agent (e.g., siRNA or shRNA) that targets a nucleic acid encoding an MS-associated TCR (e.g., a TCRA and/or TCRβ subunit) in a T cell. Expression of siRNA and shRNAs in T cells can be achieved using any conventional expression system, e.g., such as a lentiviral expression system. Expression systems for siRNA and shRNAs are described, e.g., in international application publication WO2015/142675, which is incorporated herein by reference in its entirety. Examples of shRNAs that downregulate expression of components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773, which notes that TCR expression can be inhibited using small-hairpin RNAs (shRNAs) that target nucleic acids encoding specific TCRs (e.g., TCR-α and TCR-β). By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface, the shRNA will prevent new production of TCR proteins resulting in degradation and removal of the entire TCR complex, resulting in the production of a T cell having a stable deficiency in functional TCR expression. Also see, e.g., Okada et al., Cancer Sci. 2023 Sep. 7.

In some embodiments, a subject agent includes an antisense oligonucleotide that targets (hybridizes with) an RNA and/or a DNA encoding an MS-associated TCR (e.g., a TCRA and/or TCRβ subunit), in a T cell. An antisense oligonucleotide can include one or more modified sugars, one or more modified nucleobases, one or more non-natural internucleoside linkages, or any combination thereof. Examples of non-natural internucleoside linkages include, but are not necessarily limited to: a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a, a 3′-amino phosphoramidate, an aminoalkylphosphoramidate, a phosphorodiamidate, a thionophosphoramidate, a thionoalkylphosphonate, a thionoalkylphosphotriester, a selenophosphate, and a boranophosphate. Examples of modified sugar moieties include, but are not necessarily limited to: locked nucleic acid (LNA) sugar moieties, 2′-substituted sugar moieties, 2′-O-methoxyethyl modified sugar moieties, one or more 2′-O-methyl modified sugar moieties, 2′-O-(2-methoxyethyl) modified sugar moieties, 2′-fluoro modified sugar moieties, 2′-dimethylaminooxyethoxy modified sugar moieties, and 2′-dimethylaminoethoxyethoxy modified sugar moieties. Examples of modified nucleobases include, but are not necessarily limited to: 5-methylcytosines; 5-hydroxymethyl cytosines; xanthines; hypoxanthines; 2-aminoadenines; 6-methyl derivatives of adenine; 6-methyl derivatives of guanine; 2-propyl derivatives of adenine; 2-propyl derivatives of guanine; 2-thiouracils; 2-thiothymines; 2-thiocytosines; 5-propynyl uracils; 5-propynyl cytosines; 6-azo uracils; 6-azo cytosines; 6-azo thymines; pseudouracils; 4-thiouracils; an 8-haloadenins; 8-aminoadenines; 8-thioladeninse; 8-thioalkyladenines; 8-hydroxyladenines; 8-haloguanines; 8-aminoguanines; 8-thiolguanines; 8-thioalkylguanines; 8-hydroxylguanines; 5-halouracils; 5-bromouracils; 5-trifluoromethyluracils; 5-halocytosines; 5-bromocytosines; 5-trifluoromethylcytosines; 5-substituted uracils; 5-substituted cytosines; 7-methylguanines; 7-methyladenines; 2-F-adenines; 2-amino-adenines; 8-azaguanines; 8-azaadenines; 7-deazaguanines; 7-deazaadenines; 3-deazaguanines; 3-deazaadenines; tricyclic pyrimidines; phenoxazine cytidines; phenothiazine cytidines; substituted phenoxazine cytidines; carbazole cytidines; pyridoindole cytidines; 7-deazaguanosines; 2-aminopyridines; 2-pyridones; 5-substituted pyrimidines; 6-azapyrimidines; N-2, N-6 or O-6 substituted purines; 2-aminopropyladenines; 5-propynyluracils; and 5-propynylcytosines.

Antisense oligonucleotides (ASOs) are typically small (˜15-30 nucleotides), synthetic, single-stranded nucleic acid polymers. In some instances, the ASOs comprise nucleotide modifications. Such modifications may impart useful properties, e.g. increase the biological stability of the ASOs (e.g. nucleases resistance), enhance target binding, increase tissue uptake and/or increase the physical stability of the duplex formed between the ASOs and target nucleic acids, etc.

In certain embodiments, the ASO induces steric block of a target sequence, and in such a way that it does not induce target cleavage via RNase H recruitment. For example, an ASO may comprise a chemistry which does not support RNase H cleavage (e.g., does not generate consecutive runs of DNA or DNA-like bases). For example, an ASO may comprise a “mixmer” pattern in which the ASO may comprise two or more different nucleic acid chemistries, but runs of more than 2 or 3 DNA or DNA-like bases (which would support RNase H-mediated cleavage) are avoided.

In certain embodiments, the ASO of the present disclosure may comprise DNA, RNA, and/or nucleotide analogues. The nucleotide analogues may be peptide nucleic acid (PNA), FANA, DANA, LNA, and other branched nucleic acids (ENA, cEt), phosphorodiamidate morpholino oligomer (PMO), and/or tricyclo DNA.

According to some embodiments, the ASO comprises an abasic site, i.e., the absence of a purine (adenine and guanine) or a pyrimidine (thymine, uracil and cytosine) nucleobase.

In certain embodiments, the ASO comprises a 3′ to 5′ phosphodiester (PO) linkage as naturally found in DNA or RNA. The ASO may comprise a modified internucleoside linkage, e.g. a phosphotriester linkage, a phosphorothioate (PS) linkage, a boranophosphate linkage, a phosphorodiamidate linkage, a phosphoamidate linkage, and/or a thiophosphoramidate linkage. The modified internucleoside linkage may be other modifications known in the art.

According to some embodiments, the ASO comprises one or more asymmetric centres and thus give rise to enantiomers, diasteromers, and other stereoisomeric configurations, e.g. R, S. For example, stereochemistry may be constrained at one or more modified internucleoside linkages. For example, the oligonucleotide may comprise repeated left-left-right (or SSR) chiral PS centers.

In some instances, the ASO comprises a sugar moiety as found in naturally occurring RNA (e.g., a ribofuranosyl) or a sugar moiety as found in naturally occurring DNA (e.g., a deoxyribofuranosyl). The ASO may comprise a modified sugar moiety, i.e. a substituted sugar moiety or a sugar surrogate. Substituted sugar moieties include furanosyls comprising substituents at the 2′-position, the 3′-position, the 5 ‘-position and/or the 4’-position. A substituted sugar moiety may be a bicyclic sugar moiety (BNA). Sugar surrogates include morpholino, cyclohexeynl and cyclohexitol.

The modified sugar moiety may comprise a 2′-O-methyl, 2′-O-methoxyethyl (2′-O-MOE), 2′-O-aminopropyl, 2′-deoxy, 2′-O-propyl (2′-O-AP), 2′-O-dimethylaminoethyl (2′O-DMAOE), 2′-O-dimethylaminopropyl (2′-O-DMAP), 2′-O-dimethylaminoethyloxyethyl (2′O-DMAEOE), or 2′O—N-methylacetoamido (2′O-NMA) modification or a locked or bridged ribose conformation (e.g., LNA, cEt or ENA). The modified sugar moiety may comprise other modifications known in the art.

According to some embodiments, the ASO comprises a terminal modification at its 5′ and/or 3′ end, such as a vinyl phosphonate, and/or inverted terminal bases.

In certain embodiments, the ASO comprises a nucleobase as found in naturally occurring RNA and DNA (i.e. adenine (A), thymine (T), uracil (U), guanine (G), cytosine (C), inosine (I), and 5-methyl C). The oligonucleotide may comprise a modified nucleobase, e.g. 5-hydroxymethylcytosine, 5-formylcytosine, and 5-carboxycytosine. The inclusion of 5′methylcytosine may enhance base pairing by modifying the hydrophobic nature of the oligonucleotide.

In some instances, the ASO comprises a single type of nucleic acid chemistry (e.g. full PS-MOE, or full PMO) or combinations of different nucleic acid chemistries.

For example, each of the sugar moieties in the ASO may comprise a 2′-O-methoxyethyl (2′MOE) modification and each of the internucleoside linkages may be a phosphorothioate (i.e. a fully PS-MOE oligonucleotide). PS modifications are known to result in resistance to a broad spectrum of nucleases and increase protein binding, which also improves tissue uptake. 2′MOE modifications are known to enable enhanced binding affinity to the target mRNA with minimal toxicity and reduce plasma protein binding.

According to some embodiments, the ASO comprises a combination of PO and PS internucleoside linkages. This may facilitate fine tuning of the pharmacokinetics of the oligonucleotide.

In certain embodiments, the ASO is produced using chemical synthesis and/or enzymatic ligation reactions using procedures known in the art. Alternatively, An ASO may be produced biologically using an expression vector into which the oligonucleotide is sub-cloned in an antisense orientation (e.g., RNA transcribed from the inserted oligonucleotide will be of an antisense orientation to the target nucleic acid of interest).

In some embodiments, an editing composition is provided for deleting the coding sequence or a portion thereof of human BV10, e.g., human BV10-03—the common V region of the MS-associated TCRs described herein. The human BV10-03 nucleotide sequence is provided below.

Human BV10-03 Nucleotide Sequence

| | | |
|---|---|---|
| Human | gatgctggaatcacccagagcccaagacacaag | SEQ |
| BV10- | gtcacagagacaggaacaccagtgactctgaga | ID |
| 03 | tgtcaccagactgagaaccaccgctatatgtac | NO: |
| | tggtatcgacaagacccggggcatgggctgagg | 20 |
| | ctgatccattactcatatggtgttaaagatact | |
| | gacaaaggagaagtctcagatggctatagtgtc | |
| | tctagatcaaagacagaggatttcctcctcact | |
| | ctggagtccgctaccagctcccagacatctgtg | |
| | tacttctgtgccatcagtgagtc | |

### Binding Agents

In some embodiments, a subject agent targets (e.g., specifically binds to) an MS-associated TCR (e.g., expressed by T cells), thus blocking the interaction between the TCR-expressing T cells and its target endogenous pMHC. In some cases, such an agent is an anti-TCR antibody, e.g., an antibody that specifically binds the TCRBV10 region shared among the MS-associated TCRs described herein. Anti-TCRBV10 antibodies are known and include antibody TCR1654 (ThermoFisher Scientific), antibody VER2.32.1 (Beckman Coulter Life Sciences) among others (note there is nomenclature confusion and the aforementioned antibodies specifically bind BV10-03 according to IMGT nomenclature).

In others cases, such an agent is an aptamer (e.g., a single-stranded oligonucleotide that blocks TCR-antigen binding). See. e.g., Zumrut et al., Anal Biochem. 2016 Nov. 1:512:1-7-which is an example of an aptamer developed against the TCR in Jurkat T cells. In yet other cases, such an agent is a small molecule. In some embodiments, a subject agent is a small molecule that blocks the T cell intracellular signaling cascade or blocks T cell costimulatory molecules.

Likewise, in some embodiments, a subject agent targets (specifically binds to) to an endogenous pMHC complex targeted by the MS-associated TCRs described herein, thus blocking the interaction between the MS-associated TCRs and the endogenous target pMHC. In some cases, such an agent is an autoantigen-binding RNA aptamer (e.g., a pMHC binding RNA aptamer). In other such cases, the agent is an antibody (e.g., in some cases, one lacking functional C1q binding sites in their Fc domains, e.g., Fab fragments or an antibody otherwise engineered not to bind C1q). For examples of this general approach (targeting the antigen with an antibody to block T cell activation), see, e.g., Tadano et al., Br J Cancer. 2020 October; 123 (9): 1387-1394; and Zhang et al., Proc Natl Acad Sci USA. 2014 February 18; 111 (7): 2656-61. In some cases, such an agent is a small molecule (which can, e.g., in some cases alter presentation of the peptide on the HLA. See, e.g., Li et al., J Biol Chem. 2016 Feb. 19; 291 (8): 4079-90-which uses cepharanthine in autoimmune thyroiditis. In some cases, the agent is a regulatory T cell (Treg). For example, a Treg expressing an MS-associated TCR (as described elsewhere herein), which would lead to a decrease immune response against the endogenous pMHC. In some cases, the agent is a regulatory B cell (Breg).

In some embodiments, a subject agent is a bispecific agent (e.g., a soluble bispecific agent) that includes a first moiety that binds to the endogenous pMHC (e.g., an anti-pMHC antibody), and a second moiety that agonizes an immune checkpoint molecule on the surface of the MS-associated TCRs. Moieties that agonize immune checkpoint molecules will be known to one of ordinary skill in the art, and any convenient moiety can be used. Immune checkpoint proteins include, for example: CD27, CD28, CD40, CD137 (also known as 4-1BB), ICOS, A2AR, CD47, CD160, B7H3, B7H4, CD40, CD40L, SIRP alpha, TIGIT, BTLA, CTLA-4, LAG3, TIM3, VISTA, PD-1, PD-L1. In some cases, the second moiety is a PD-1 agonist. In some cases, the bispecific agent (e.g., soluble bispecific agent) is the PD-1 agonist immune modulating monoclonal TCR against autoimmunity (ImmTAAI).

The phrases “specifically binds”, “specific for”, “immunoreactive” and “immunoreactivity”, and “antigen binding specificity”, when referring to a binding agent, refer to a binding reaction with an antigen which is highly preferential to the antigen or a fragment thereof, so as to be determinative of the presence of the antigen in the presence of a heterogeneous population of antigens (e.g., proteins and other biologics, e.g., in a sample). Thus, under designated immunoassay conditions, the specified agent binds to a particular antigen and does not bind in a significant amount to other antigens present in the sample.

In some embodiments, an agent of the present disclosure “specifically binds” its target if it binds to or associates with the target with an affinity or Kₐ (that is, an equilibrium association constant of a particular binding interaction with units of 1/M) of, for example, greater than or equal to about 10⁵ M¹. In certain embodiments, the agent binds to the target with a Kₐ greater than or equal to about 10⁶ M¹, 10⁷ M¹, 10⁸ M¹, 10⁹ M¹, 10¹⁰ M¹, 10¹¹ M¹, 10¹² M¹, or 10¹³ M¹. “High affinity” binding refers to binding with a Kₐ of at least 10⁷ M¹, at least 10⁸ M¹, at least 10⁹ M¹, at least 10¹⁰ M¹, at least 10¹¹ M⁻¹, at least 10¹² M⁻¹, at least 10¹³ M¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the agent binds to the target with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the agent for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

### Targeted Protein Degradation (TPD)

In some embodiments, a subject agent targets an MS-associated TCR for degradation. For example, in some cases, such an agent is a PROteolysis TArgeting Chimera (PROTAC) that targets an MS-associated TCR for degradation. PROTACs are heterobifunctional small molecules having two ligands joined by a linker: one ligand recruits and binds a protein of interest (POI) (e.g., MS-associated TCR) while the other recruits and binds an E3 ubiquitin ligase. Simultaneous binding of the POI and ligase by the PROTAC induces ubiquitylation of the POI and its subsequent degradation by the ubiquitin-proteasome system (UPS), after which the PROTAC is recycled to target another copy of the POI. Thus, a PROTAC has a catalytic-type mechanism of action. For more details, see, e.g., Békés et al., Nat Rev Drug Discov. 2022 March; 21 (3): 181-200.

As another example, in some cases, a subject agent is a molecular glue (which mediates proximity-induced protein degradation) that targets an MS-associated TCR for degradation. Molecular glues are monovalent small molecules (<500 Da) that reshape the surface of an E3 ligase receptor, promoting novel protein-protein interactions (PPIs). In contrast to the original PROTACs, in which two ligands are connected by a flexible linker that can twist and turn and allow the two proteins to form contacts, molecular glues were believed to more directly enhance complex formation between an E3 ligase and a target protein by squeezing between protein-protein interfaces and are generally defined as small molecules that interact with two protein surfaces to induce or enhance the affinity of these two proteins for each other. For more details, see, e.g., Dong et al., J Med Chem. 2021 Aug. 12; 64 (15): 10606-10620; and Sasso et al., Biochemistry. 2023 Feb. 7; 62 (3): 601-623.

### Vaccine Against TCR

In some embodiments, a subject agent is a vaccine against an MS-associated TCR. Such a vaccine triggers immunity against the MS-associated TCR. In some cases, the agent is a protein fragment of an MS-associated TCR (e.g., a TCRBV10 region or a CDR region). In some cases, the agent is a nucleic acid encoding an MS-associated TCR or a fragment thereof (e.g., a TCRBV10 region or a CDR region). As an illustrative example, in some cases a subject agent is an mRNA (e.g., in vitro transcribed or synthesized mRNA) encoding a TCRBV10 region or a CDR region (e.g., a CDR3 region) of an MS-associated TCR. See, e.g., Tusup et al., Pharmaceutics. 2021 Jul. 7; 13 (7): 1040. “mRNA-Based Anti-TCR CDR3.

A subject vaccine against an MS-associated TCR can include a portion of an MS-associated TCR (e.g., a TCRBV10 region or a CDR region, a fragment of a CDR, a CDR plus some surrounding amino acid sequence, and the like), or can be a nucleic acid that encodes such a portion, of any convenient length, in some cases having a length in a range of from 5-30 amino acids (e.g., 5-25, 5-20, 5-18, 5-17, 5-15, 5-10, 7-30, 7-25, 7-20, 7-18, 7-17, 7-15, 7-10, 8-30, 8-25, 8-20, 8-18, 8-17, 8-15, 8-10, 9-30, 9-25, 9-20, 9-18, 9-17, 9-15, 10-30, 10-25, 10-20, 10-18, 10-17, 10-15, 20-30, or 25-30 amino acids). In some cases, the portion of the MS-associated TCR has a length in a range of from 7-20 amino acids (e.g., 7-15, 7-11, 7-10, 8-20, 8-15, 8-10, 15-19, 16-18, or 9-17 amino acids). In some cases, a subject peptide-based vaccine has a length in a range of from 8-10 amino acids. In some cases, a subject peptide-based vaccine has a length in a range of from 16-18 amino acids. In some cases, a subject peptide-based vaccine has a length of about 10 amino acids. In some cases, a subject peptide-based vaccine has a length of about 15 amino acids.

### Tolerizing Vaccine Against Autoantigen

In some embodiments, a subject agent is a tolerizing vaccine against the autoantigen (e.g., endogenous pMHC), i.e., the agent elicits autoantigen-specific immune tolerance. Tolerogenic (tolerizing) vaccines deliver antigens with the purpose of suppressing immune responses (e.g., induce or increase a suppressive immune response) and promoting robust long-term antigen-specific immune tolerance.

Because immune tolerance is orchestrated by APCs, numerous tolerogenic vaccine platforms have been developed to deliver autoantigens to specific APC subtypes. Some of these tolerogenic vaccine platforms include protein/peptide-, nanoparticle-, and DNA/RNA-based vaccines. Furthermore, immunosuppressive cell types such as tDCs, have been manipulated and expanded ex vivo and reintroduced as cell-based tolerogenic vaccines. Together, these agents can be summarized as antigen-specific tolerogenic vaccines.

In some embodiments, a subject agent is a peptide-based vaccine (e.g., administration of a peptide or pMHC as described herein can induce antigen-specific tolerance). In other words, administration, e.g., injection, of the peptide or a fragment thereof can tolerize the immune system to the presence of the antigen. See, e.g., Peptide- and Protein-Based Vaccines as discussed in Moorman et al., Front Immunol. 2021 Mar. 29:12:657768. Peptide vaccines are typically administered repeatedly and often require high doses. Thus, in some cases, a protein carrier is used to increase the stability, half-life, and bioavailability of autoantigen peptides to increase the efficacy of peptide-based tolerogenic vaccines. Protein carriers such as mAb, cytokines, cells, and pathogen derived immunosuppressive or adhesion proteins have served as targeting moieties to introduce tethered peptides into specific immunological niches or as tolerogenic adjuvants to favor tolerance, and such protein carriers can be used with a subject agent. Nanoparticles (NP) and microparticles (MP) can also be used as carriers. In some cases, a peptide based vaccine is administered with a dendritic cell (DC)-targeting carrier protein. For more details, see, e.g., Moorman et al., Front Immunol. 2021 Mar. 29:12:657768. In some cases, a subject agent induces immunological tolerance to a presented HLA-peptide complex. In some cases, the agent is a tolerogenic vaccine. In some cases, the agent induces tolerogenic dendritic cells. In some cases, a subject agent employs tolerogenic peptide-carrier vaccine targeting, and in some cases, a subject agent employs a tolerogenic adjuvant strategy. For more details, see, e.g., Moorman et al., Front Immunol. 2021 Mar. 29:12:657768.

### Agents for Induction of Tregs

In some embodiments, a subject agent reprograms T cells expressing the MS-associated TCRs to differentiate and expand into regulatory T cells (Tregs). For example, in some embodiments, the agent is a Navacim. Navacims find specific disease-causing autoantigen-experienced T cells and present a high-density array of disease specific pMHC to cognate T cell receptors (TCR). Navacim high density binding to TCR micro-clusters causes signaling which reprograms disease-causing T cells (effectors) to differentiate and expand into disease-regulating Treg cells (suppressors). The disease-specific Treg cells selectively suppress autoimmune attacks on self in the diseased organ. Navacims are nanoparticles coated with pMHC complexes (e.g., in some cases made with an iron oxide nanoparticle core encapsulated with a polymer coating conjugated to the pMHC complex). Navacims become pharmacologically active and organ-specific from the unique antigenic peptide selected for each Navacim. Navacims induce expansion of disease-specific Tregs, and it is this expanded population of Tregs that in turn activate other immune regulatory cells (including B cells) to broadly shut down the activity of polyclonal inflammatory cells contributing to disease.

### Gene Therapy

In some embodiments, a subject agent provides for an increased blood brain barrier (BBB) integrity by increasing the amount of a ZO protein (e.g., ZO-1). In some such cases, a subject agent is a nucleic acid encoding ZO-1 or a fragment of ZO-1 (e.g., RNA or DNA).

In some embodiments, the amino acid sequence encoded includes TAIWEQHTV (SEQ ID NO: 7). In some cases, the amino acid sequence encoded includes an amino acid sequence having 3 mutations (e.g., substitution, insertion, deletion) or less (e.g., 2 mutations, 1 mutation, or no mutations) relative to SEQ ID NO: 7. In some cases, the amino acid sequence encoded includes the amino acid sequence MEETAIWEQHTVTLHRA (SEQ ID NO: 6). In some cases, the amino acid sequence encoded includes an amino acid sequence having 3 mutations (e.g., substitution, insertion, deletion) or less (e.g., 2 mutations, 1 mutation, or no mutations) relative to SEQ ID NO: 6. The amino acid sequence encoded can be any convenient length, in some cases having a length in a range of from 5-30 amino acids (e.g., 5-25, 5-20, 5-18, 5-17, 5-15, 7-30, 7-25, 7-20, 7-18, 7-17, 7-15, 8-30, 8-25, 8-20, 8-18, 8-17, 8-15, 9-30, 9-25, 9-20, 9-18, 9-17, 9-15, 10-30, 10-25, 10-20, 10-18, 10-17, or 10-15 amino acids). In some cases, the amino acid sequence encoded has a length in a range of from 7-20 amino acids (e.g., 7-11, 8-10, 15-19, 16-18, or 9-17 amino acids). In some cases, the amino acid sequence encoded has a length in a range of from 8-10 amino acids. In some cases, the amino acid sequence encoded has a length in a range of from 16-18 amino acids. In some cases, the amino acid sequence encoded has a length of about 9 amino acids. In some cases the amino acid sequence encoded has a length of about 17 amino acids.

In some cases, the nucleic acid includes a nucleotide sequence that (i) is operably linked to a promoter (e.g., constitutive or inducible promoter), and (ii) encodes a ZO-1 peptide. For information regarding ZO-1 and its role in the BBB, see, e.g., Olsson et al., Mult Scler Relat Disord. 2021 September: 54:103136; Kirk et al., J Pathol. 2003 October; 201 (2): 319-27; and Bennett et al., J Neuroimmunol. 2010 Dec. 15; 229 (1-2): 180-91.

### Compositions

The present disclosure also provides compositions. In certain embodiments, the compositions find use, e.g., in practicing the methods of the present disclosure.

According to some embodiments, a composition of the present disclosure includes any of the agents of the present disclosure. For example, the agent may be any of the agents described in the Agents for Preventing or Inhibiting MS-Associated Adaptive Immune Responses section hereinabove, which is incorporated but not reiterated herein for purposes of brevity. According to some embodiments, a composition of the present disclosure includes any of the pMHCs or multimers thereof, or agents (e.g., nanoparticles, etc.) comprising the same.

In certain aspects, a composition of the present disclosure includes the agent present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure include compositions suitable for administration to a subject in need thereof. In some embodiments, a composition of the present disclosure includes any of the agents of the present disclosure, and a pharmaceutically acceptable carrier.

An agent of the present disclosure can be incorporated into a variety of formulations for therapeutic administration. More particularly, the agent can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agents for administration to the individual (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the agent against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the agent, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Therapeutic Methods

Provided are methods of preventing or inhibiting an MS-associated adaptive immune response in a subject in need thereof. In some embodiments, the methods comprise administering an agent of the present disclosure to the subject. In certain instances, the methods are for treating multiple sclerosis in a subject in need thereof.

In some embodiments, provided are methods of preventing or inhibiting a multiple sclerosis (MS)-associated adaptive immune response in a subject in need thereof, the method comprising administering to the subject any of the soluble pMHCs or pMHC multimers of the present disclosure, or any other agents comprising such pMHCs or pMHC multimers, in an amount effective to prevent or inhibit the MS-associated adaptive immune response in the subject. In certain embodiments, such methods are for treating multiple sclerosis in a subject in need thereof.

Also provided are methods of preventing or inhibiting a multiple sclerosis (MS)-associated adaptive immune response in a subject in need thereof, the method comprising administering to the subject a therapeutically effective amount of an antibody that specifically binds a TCRBV10 region. In certain embodiments, such methods are for treating multiple sclerosis in a subject in need thereof. Antibodies that specifically bind TCRBV10 are described elsewhere herein. In some instances, the antibody is chosen from an IgG, single chain Fv (scFv), Fab, (Fab)₂, (scFv′)₂, or a single variable domain located on a heavy chain (VHH). Depletion of disease-associated T cells using an antibody that binds to a V region of the TCR expressed by the disease-associated T cells is known and described, e.g., in Britanova et al. (2023) “Targeted depletion of TRBV9+ T cells as immunotherapy in a patient with ankylosing spondylitis” Nature Medicine 29:2731-2736.

In some embodiments, the antibody is the antigen binding domain of an engineered cell surface receptor, and the method comprises administering a therapeutically effective amount of a population of immune cells expressing the engineered cell surface receptor to the subject. A non-limiting example of an engineered cell surface receptor is a chimeric antigen receptor (CAR). The immune cells may be, e.g., T cells, natural killer (NK) cells, or the like.

Also provided are methods of preventing or inhibiting a multiple sclerosis (MS)-associated adaptive immune response in a subject in need thereof, the method comprising administering to the subject a therapeutically effective amount of immune regulatory cells expressing an MS-associated TCR described herein. In some instances, such methods are for treating multiple sclerosis in a subject in need thereof.

According to any of the therapeutic methods of the present disclosure, the subject may be HLA-DRB1*15 positive. For example, prior to the administering, the subject may have been identified as HLA-DRB1*15 positive.

According to any of the therapeutic methods of the present disclosure, the subject has been identified as having, or being at risk of developing MS. In some cases, the subject has MS. In some cases, the subject has RRMS. In some cases, the subject has SPMS. In some cases, the subject has PPMS. In some cases, the subject has PRMS. In some cases, the subject has Marburg variant multiple sclerosis. In some cases, the subject has tumefactive multiple sclerosis. In some cases, the subject has neuromyelitis optica (Devic's disease). In some cases, the subject has Balo's concentric sclerosis.

By “treatment” it is meant that at least an amelioration of one or more symptoms associated with a condition afflicting the subject is achieved, where amelioration is used in a broad sense to refer to at least a reduction in the magnitude of a parameter, e.g., a symptom associated with the condition being treated. As such, treatment also includes situations where a pathological condition, or at least symptoms associated therewith, are completely inhibited, e.g., prevented from happening, or stopped, e.g., terminated, such that the adult mammal no longer suffers from the condition, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

For administration purposed, those of skill in the art will readily appreciate that dose levels of a subject agent can vary as a function of the specific agent, the nature of the delivery vehicle, and the like. Preferred dosages for a given compound are readily determinable by those of skill in the art by a variety of means.

An “effective amount” or “effective dose” refers to that amount which is capable of having the desired effect, e.g., ameliorating or delaying progression of the diseased, degenerative or damaged condition, and the like. An effective amount can be determined on an individual basis and will be based, in part, on consideration of the symptoms to be treated and results sought. A “therapeutically effective dose” or “therapeutic dose” or “therapeutically effective amount” or “therapeutic amount” is an amount sufficient to effect desired clinical results (i.e., achieve therapeutic efficacy). A therapeutically effective dose can be administered in one or more administrations. An effective amount (e.g., therapeutically effective amount) can be determined by one of ordinary skill in the art employing such factors.

In some cases, an effective amount of a subject agent will reduce MS and/or symptoms of MS. In some cases, progression of MS will be blocked and in some cases MS will be reduced. An effective dose (e.g., therapeutically effective dose) can be expected to be in a range of from about 0.001 to about 100 mg/kg body weight per day (for humans), in some cases from about 0.1 to about 50 mg/kg body weight per day, in some cases from about 1 to about 50 mg/kg body weight per day, in some cases about 5 to about 40 mg/kg body weight per day, in some cases about 2 to about 15 mg/kg body weight per day, and in some cases about 25 to about 40 mg/kg bodyweight per day. In some cases, an effective does is in a range of from 5 to 100 mg/kg bodyweight per day (e.g., 5-80, 5-50, 5-40, 5-30, 5-20, 5-10, 8-100, 8-80, 8-50, 8-40, 8-30, 8-20, 10-100, 10-80, 10-50, 10-40, 10-30, or 10-20 mg/kg bodyweight per day). In some cases, an effective does is in a range of from 8 to 100 mg/kg bodyweight per day. In some cases, an effective does is about 8 mg/kg bodyweight per day.

Dosage and frequency may vary depending on the half-life of the agent. It will be understood by one of skill in the art that such guidelines will be adjusted for the molecular weight of the active agent. The dosage may also be varied for localized administration, e.g. intranasal, inhalation, etc., or for systemic administration, e.g. i.m., i.p., i.v., s.c., and the like.

The treatment course may be less than about 12 weeks, less than about 8 weeks, less than about 4 weeks, and may be, for example, from 1-12 weeks, from 2-12 weeks, from 4-12 weeks, from 4-8 weeks, etc. Administration may be once a week, twice a week, every other day, daily, twice a day, every two weeks, etc., and in some embodiments is once a week. In some embodiments, more than one course of treatment is administered. In some cases, the course of treatment is in a range of from 1-24 weeks (e.g., from 1-8 weeks, 1-4 weeks, about 1 week, about 2 weeks, or about 3 weeks). In some cases, the course of treatment is in a range of from 1 day-8 weeks (e.g., from 1 day-6 weeks, 1 day-4 weeks, 1 day-2 weeks, 1 day-7 days, 3 days-8 weeks, 3 days-6 weeks, 3 days-4 weeks, 3 days-2 weeks, or 3 days-7 days). In some cases, the compound is administered for 2 or more days (e.g., 3 or more, 4 or more, 5 or more, 6 or more, 7 or more days, 14 or more days, or 1 month or more). In some cases, the compound is administered for 3 or more days.

A subject agent can be administered to an individual by any route of administration appreciated in the art, including but not limited to oral administration, administration by injection (specific embodiments of which include intravenous, subcutaneous, intraperitoneal or intramuscular injection), administration by inhalation, intranasal, or topical administration, either alone or in combination with other agents designed to assist in the treatment of the individual. The route of administration should be determined based on a number of considerations appreciated by the skilled artisan including, but not limited to, the desired physiochemical characteristics of the treatment. Treatment may be provided for example, 2-8° C. or higher, while also making the formulation useful for parenteral injection. As appropriate, preservatives, stabilizers, buffers, antioxidants and/or other additives may be included. The formulations may contain a divalent cation (including but not limited to MgCl₂, CaCl2, and MnCl2) and/or a non-ionic surfactant (including but not limited to Polysorbate-80 (TWEEN 80™), Polysorbate-60 (TWEEN 60™), Polysorbate-40 (TWEEN 40™), and Polysorbate-20 (TWEEN 20™) polyoxyethylene alkyl ethers, including but not limited to BRIJ 58™, BRIJ 35™, as well as others such as TRITONX-100™, TRITONX-114™, NP40™, Span 85 and the PLURONIC® series of non-ionic surfactants (e.g., PLURONIC® 121). Any combination of such components form specific embodiments of the present disclosure.

“Pharmaceutically acceptable salts” and “pharmaceutically acceptable esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “co-administration”, “co-administer”, and “in combination with” include the administration of two or more therapeutic agents (e.g., two or more subject agents described herein, a subject agent described herein and an agent known for MS treatment such as a corticosteroid, e.g., methylprednisolone, and the like) either simultaneously, concurrently or sequentially within no specific time limits. A subject agent may be co-administered with any number of agents-such as agents used in the treatment of MS. Such agents may include, for example, beta interferons, Glatiramer (Copaxone, Glatopa), Cladribine (Mavenclad), Dimethyl fumarate (Tecfidera), Fingolimod (Gilenya), Monomethyl fumarate (Bafiertam), Ofatumumab (Kesimpta), Ozanimod (Zeposia), Ponesimod (Ponvory), Siponimod (Mayzent), Teriflunomide (Aubagio), Alemtuzumab (Lemtrada), mitoxantrone (Novantrone), Natalizumab (Tysabri), ocrelizumab (Ocrevus), Methylprednisolone (Solu-Medrol), Prednisone (Deltasone), ACTH (H.P. Acthar Gel), muscle relaxants like baclofen (Lioresal) and tizanidine (Zanaflex), Sedatives like clonazepam (Klonopin) and diazepam (Valium), Amantadine (Symmetrel), Armodafinil (Nuvigil), Modafinil (Provigil), Oxybutynin (Ditropan), tolterodine (Detrol), bupropion (Wellbutrin), fluoxetine (Prozac), sertraline (Zoloft), and the like.

In one embodiment, the agents are present in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic.

For purposes of completeness, non-limiting aspects and embodiments of the present disclosure are further disclosed in the following numbered clauses.

1. A soluble peptide-major histocompatibility complex (pMHC), wherein the MHC is HLA-DRB1*15 and the peptide comprises, consists essentially of, or consists of an amino acid sequence chosen from TAIWEQHTV (SEQ ID NO: 7), IALWESHDV (SEQ ID NO:9), VAIKEAHDI (SEQ ID NO: 11), IGLAESHDN (SEQ ID NO: 13), ELIWEQYTV (SEQ ID NO: 15), LTIWEQHTA (SEQ ID NO: 17), and LAVMESHAI (SEQ ID NO: 19).

2. A soluble pMHC multimer comprising two or more of the pMHC of clause 1.

3. The soluble pMHC multimer of clause 2, which is a pMHC dimer, tetramer, or pentamer.

4. The soluble pMHC multimer of clause 2, wherein the pMHC is multimerized using a polymer backbone.

5. The soluble pMHC multimer of clause 4, wherein the pMHC monomers is multimerized using a dextran backbone.

6. The soluble pMHC or pMHC multimer of any one of clauses 1-5, conjugated or fused to a T cell inhibitor.

7. The soluble pMHC or pMHC multimer of clause 6, wherein the T cell inhibitor is an agonist of an immune checkpoint molecule.

8. The soluble pMHC or pMHC multimer of clause 7, wherein the immune checkpoint molecule is chosen from programmed cell death-1 (PD-1), cytotoxic T-lymphocyte-associated antigen 4 (CTLA-4), T cell immunoreceptor with Ig and ITIM domains (TIGIT), T-cell immunoglobulin domain and mucin domain 3 (TIM-3), V-domain Ig suppressor of T cell activation (VISTA), and B and T lymphocyte attenuator (BTLA).

9. The soluble pMHC or pMHC multimer of clause 6, wherein the T cell inhibitor is an anti-CD3 binding agent.

10. The soluble pMHC or pMHC multimer of clause 9, wherein the anti-CD3 binding moiety is an anti-CD3 antibody.

11. The soluble pMHC or pMHC multimer of any one of clauses 1-10, conjugated to an agent.

12. The soluble pMHC or pMHC multimer of clause 11, wherein the agent is chosen from a toxin, a radioisotope, a radiation sensitizing agent, a detectable label, a half-life extending moiety, and any combination thereof.

13. A nanoparticle conjugated to one or more of the soluble pMHC or pMHC multimer of any one of clauses 1-12.

14. A composition comprising the soluble pMHC or pMHC multimer of any one of clauses 1-11 or the nanoparticle of clause 13.

15. The composition of clause 14, wherein the composition is suitable for administration to a subject.

16. A method of preventing or inhibiting a multiple sclerosis (MS)-associated adaptive immune response in a subject in need thereof, the method comprising administering to the subject the soluble pMHC or pMHC multimer of any one of clauses 1-11 or the nanoparticle of clause 13 in an amount effective to prevent or inhibit the MS-associated adaptive immune response in the subject.

17. A method of treating multiple sclerosis in a subject in need thereof, the method comprising administering to the subject a therapeutically effective amount of the soluble pMHC or pMHC multimer of any one of clauses 1-11 or the nanoparticle of clause 13.

18. A method of preventing or inhibiting a multiple sclerosis (MS)-associated adaptive immune response in a subject in need thereof, the method comprising administering to the subject a therapeutically effective amount of an antibody that specifically binds a TCRBV10 region.

19. A method of treating multiple sclerosis in a subject in need thereof, the method comprising administering to the subject a therapeutically effective amount of an antibody that specifically binds a TCRBV10 region.

20. The method of clause 19, wherein the antibody is chosen from an IgG, single chain Fv (scFv), Fab, (Fab)₂, (scFv′)₂, or a single variable domain located on a heavy chain (VHH).

21. The method of clause 19, wherein the antibody is the antigen binding domain of an engineered cell surface receptor, and wherein the method comprises administering a therapeutically effective amount of a population of immune cells expressing the engineered cell surface receptor to the subject.

22. The method of clause 21, wherein the engineered cell surface receptor is a chimeric antigen receptor (CAR).

23. The method of clause 21 or 22, wherein the immune cells are T cells.

24. The method of clause 21 or 22, wherein the immune cells are natural killer (NK) cells.

25. A genetically modified cell that expresses a T cell receptor (TCR) comprising a TCRβ chain comprising a TCRβ CDR3 sequence of one of SEQ ID NOs: 1-3.

26. The cell of clause 25, wherein the TCRβ CDR3 sequence is CAISESWTGGSDTQYF (SEQ ID NO: 1) and the TCRα CDR3 sequence is CIVRPNTGTASKLTF (SEQ ID NO: 4).

27. The cell of clause 25, wherein the TCRβ CDR3 sequence is CAISESWAGGTDTQYF (SEQ ID NO: 2) and the TCRα CDR3 sequence is CIVRGNTGTASKLTF (SEQ ID NO: 5).

28. The cell of clause 25, wherein the TCRβ CDR3 sequence is CAISEGWTGNTDTQYF (SEQ ID NO: 3) and the TCRα CDR3 sequence is CIVRGNTGTASKLTF (SEQ ID NO: 5).

29. The cell of any one of clauses 25-28, wherein the cell is an immune regulatory cell.

30. The cell of clause 29, wherein the immune regulatory cell is a regulatory T cell (Treg).

31. A method of preventing or inhibiting a multiple sclerosis (MS)-associated adaptive immune response in a subject in need thereof, the method comprising administering to the subject a therapeutically effective amount of immune regulatory cells as defined in clause 29 or 30.

32. A method of treating multiple sclerosis in a subject in need thereof, the method comprising administering to the subject a therapeutically effective amount of immune regulatory cells as defined in clause 29 or 30.

33. The method of any one of clauses 17-24, 31 or 32, wherein the MS is relapsing-remitting MS (RRMS), secondary-progressive MS (SPMS), primary-progressive MS (PPMS), progression-relapsing MS (PRMS), Marburg variant multiple sclerosis, tumefactive multiple sclerosis, neuromyelitis optica (Devic's disease), or Balo's concentric sclerosis.

34. The method of any one of clauses 17-24 or 31-33, wherein prior to the administering, the subject has been identified as HLA-DRB1*15 positive.

35. A nucleic acid adapted to specifically reduce expression by RNA interference (RNAi) of a TCRβ chain comprising a TCRBV10.3 region.

36. A nucleic acid adapted to specifically reduce expression by RNA interference (RNAi) of a TCRβ chain comprising a TCRβ CDR3 sequence chosen from CAISESWTGGSDTQYF (SEQ ID NO: 1), CAISESWAGGTDTQYF (SEQ ID NO: 2), CAISEGWTGNTDTQYF (SEQ ID NO: 3), or any combination thereof.

37. A nucleic acid adapted to specifically reduce expression by RNA interference (RNAi) of a TCRβ chain comprising a TCRα CDR3 sequence chosen from CIVRPNTGTASKLTF (SEQ ID NO: 4), CIVRGNTGTASKLTF (SEQ ID NO: 5), or both.

38. The nucleic acid of any one of clauses 35-37, wherein the nucleic acid is a short interfering RNA (siRNA), a short hairpin RNA (shRNA), a DROSHA substrate, a Dicer substrate, a microRNA (miRNA), or a PIWI-interacting RNA (piRNA).

39. The nucleic acid of any one of clauses 35-37, wherein the nucleic acid is an siRNA.

40. The nucleic acid of any one of clauses 35-37, wherein the nucleic acid is an antisense oligonucleotide (ASO).

41. A virus particle, liposome or nanoparticle comprising an expression construct comprising the nucleic acid of any one of clauses 35-40.

42. A method of delivering an expression construct comprising the nucleic acid of any one of clauses 35-40 to T cells in a subject in need thereof, the method comprising administering the virus particle, liposome or nanoparticle to a subject in need thereof, wherein the expression construct is delivered to T cells in the subject.

43. The method of clause 42, wherein prior to the administering, the subject has been identified as HLA-DRB1*15 positive.

The following examples are offered by way of illustration and not by way of limitation.

### EXPERIMENTAL

### Example 1—Identification of ZO-1 as a Target of the Adaptive Immune System in Multiple Sclerosis (MS)

A fraction of the human cellular immune response includes T cells with “public” T cell receptors (TCRs). When multiple people present the same antigen with the same HLA allele, many of the responding T cells encode the exact same TCR making it “public”. These public TCRs have been observed in the immune response to many pathogens. Described in this example is the identification of multiple sclerosis (MS)-specific TCRs, followed by identification of the antigen to which those TCRs bind in MS patients with HLA DRB1-1501.

To identify MS-specific TCRs, a case/control study was performed consisting of thousands of patients with MS and many thousands of HLA matched controls without MS. First, the TCR beta chain (TCRB) repertoire was sequenced from the blood of cases and controls. Using statistical methods, the TCR sequences that are enriched in the cases versus the controls were identified. Many of these TCRs clustered into groups, where the clusters are defined as having nearly identical TCRβ sequences. The hypothesis is that all TCRs in the same cluster bind to the same HLA presented antigen. The HLA presenting the antigen can be readily deduced by statistical correlation with the members of a given cluster. The missing piece is the antigen that the HLA is presenting.

In the case of MS patients with HLA DRB1-1501, a primary cluster that includes 3 TCRβ sequences that are nearly identical (Hamming distance 3) were identified:

| | TCRB CDR3 Sequence | SEQ ID NO |
|---|---|---|
| TCR1 | CAISESWTGGSDTQYF | 1 |
| TCR2 | CAISESWAGGTDTQYF | 2 |
| TCR3 | CAISEGWTGNTDTQYF | 3 |

Next, the TCR alpha chain (TCRA) sequences that pair with the identified TCRβ sequences. From the blood of 47 MS patients with DRB1-1501, the pairSEQ® assay (Howie et al. (2015) Sci Transl Med. 7 (301): 301ra131.) was performed to determine the fully paired TCR alpha and beta chains. The paired TCRA sequences also clustered with very similar amino acid sequences, providing further evidence that the TCRs are targeting the same HLA presented antigen:

| | TCRA CDR3 Sequence | SEQ ID NO |
|---|---|---|
| TCR1 | CIVRPNTGTASKLTF | 4 |
| TCR2 | CIVRGNTGTASKLTF | 5 |
| TCR3 | CIVRGNTGTASKLTF | 5 |

In order to identify the antigen of interest, a “de-orphanization” technique was developed and applied for each TCR. This technique broadly searches the antigen space. A positional scanning combinatorial peptide library (PS-CPL) was designed for the base peptide, GGIxxVxSxxVG, where x represents an equal mixture of all 20 natural occurring amino acids.

A combinatorial peptide library was created by introducing each naturally occurring amino acid, one at a time at each position from position 3 to 11. TCRa^{-/-}TCRb^{-/-} jurkat cells expressing a TCR-of-interest were co-cultured with HLA-DRB1*15:01-expressing K562 cells loaded with the combinatorial peptide library for 2 hours and assessed for CD69 upregulation.

A combinatorial library yielded 180 wells of peptides. As a negative control, 40 wells with no peptide were included. These test peptides and negative controls were tested using three 96-well plates. For quality control, negative controls were distributed evenly within each 96-well plate. To ensure there is no well-effect, the correlation between the negative controls and the order in which data was read from a NovoCyte flow cytometer system was first estimated. Significant (p-value <0.05) correlation resulted in the correction of a given plate's data using the slope coefficient. To correct for potential variation in CD69 activity measurements between plates, MFI values were normalized within each plate using the following equation:

$${x}^{′} = ( x - {s}_{neg} ) / {m}_{neg}$$

where x is the corrected MFI value, m_{neg} is the average corrected MFI of the negative controls, and s_{neg} is the standard deviation of the negative controls. Following normalization, data from all plates was combined for subsequent analysis.

A position weight matrix was created from normalized MFI values, with a single value per amino acid/position. Using this PWM, a score was then assigned to every possible 9-mer in proteomes of interest. Scores are simply the sum of each amino acid/position normalized MFI. For this step, both human and viral proteomes were utilized. The human 9-mer database was constructed from the reference proteome (GRCh38) as well as 44 proteomes from the Human Pangenome Reference Consortium, all of which are available on Ensembl (uswest.ensembl.org/index.html). The viral database was constructed by downloading all viral proteomes with human hosts available on UniProt, as well as all available EBV and HHV6a amino acid sequences from NCBI. The latter two species were chosen based on previous implication of their involvement in multiple sclerosis.

Benchmark TCRs consistently showed that the correct peptide is within the top 3 highest ranked peptides. Therefore, the top 10 peptides were selected for synthesis and characterization in the lab.

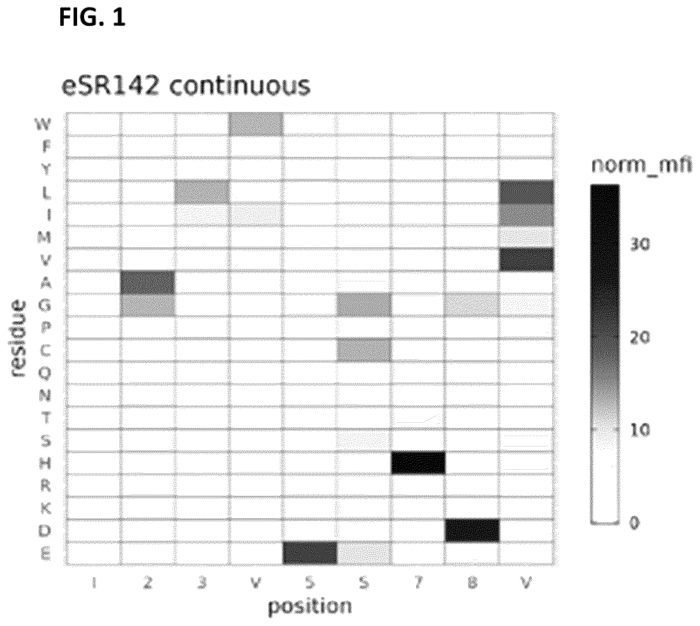

Shown in FIG. 1 are normalized median CD69 expression levels by TCR1-expressing cells co-stimulated with peptide containing a fixed amino acid at each position.

Using a peptide score, whereby the normalized CD69 MFI for each amino acid at each position is added, a list of 50 peptides from the human proteome were nominated.

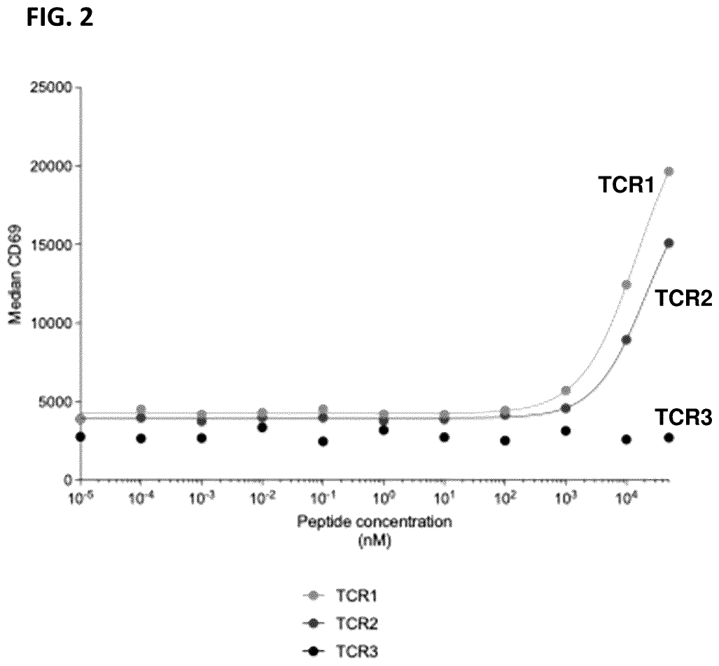

TCR-of-interest-expressing cells were co-stimulated with HLA-DRB1*15:01-expressing K562s loaded with peptide at the concentrations of 50⁴, 10⁴, 10³, 10², 10¹, 10⁰, 10⁻¹, 10⁻², 10⁻³, 10⁻⁴, 10⁻⁵ nM. Shown in FIG. 2 is dose response of TCRs to the peptide MEETAIWEQHTVTLHRA (SEQ ID NO: 6). TCR1 is MS_DR15_ESg1_2 (an MS-associated TCR). TCR2 is MS_DR15_ESg1_Mun1 (an MS-associated TCR). TCR3 is MS_DR15_ESg1_Mun1e1 (an MS-associated TCR and negative control).

The de-orphanization process resulted in a single antigen target in the human genome. From the peptide sequence (MEETAIWEQHTVTLHRA-SEQ ID NO: 6), it was deduced that the target is the Zona Occludens 1 (ZO-1) protein encoded by the gene TJP1.

| | | SEQ | | SEQ |
|---|---|---|---|---|
| Peptide | Peptide | ID | | ID |
| Name | Sequence | NO | 9mer | NO |
| ZO-1 | MEETAIWEQHTVTLHRA | 6 | TAIWEQHTV | 7 |

Since ZO-1 plays a crucial role in the structure of the blood-barrier, it is reasonably expected with the benefit of the present disclosure that a crucial (likely causative) auto-reaction in MS patients is the direct attack on the blood-brain barrier (BBB) which then allows multiple cells from the adaptive immune system through the BBB and into the brain. The downstream impact is inflammation that impacts myelin generating cells and leads to the plaques/damage resulting in MS symptoms. The attack is likely a B cell immune response to ZO-1 directed by the CD-4 helper cells that specifically bind the DRB1-1501 presented epitope TAIWEQHTV.

### Example 2—Validation of ZO-1 as a Candidate Target Antigen in MS

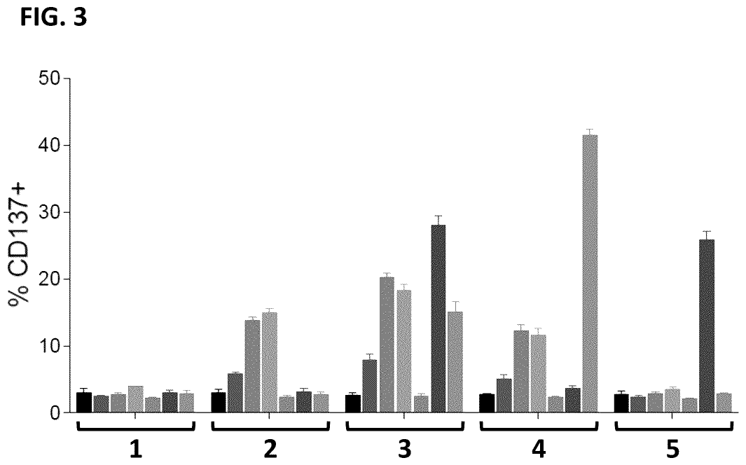

In this example, TCRs were delivered to primary human T cells via transfection of IVT RNA. Antigen-encoding constructs were delivered to K562 ‘target’ cells that are HLA-DRB1*15:01 positive also via transfection of IVT RNA. T cells and target cells were then co-cultured overnight, followed by measurement of T cell activation via CD137 expression to each of the antigen-encoding constructs. Data is shown in FIG. 3. Antigen encoding constructs (1)-(5) were as follows: (1) Mock; (2) ZO-1; (3) ZO-1_MBP; (4) ZO-1_COVID; and (5) MBP.

For each condition (antigen encoding constructs (1)-(5)), the bars from left to right indicate T cell activation via CD137 expression from the following TCRs: (a) mock; (b) MS_DR15_ESg1_Mun1 (an MS-associated TCR); (c) MS_DR15_ESg1_2 (an MS-associated TCR); (d) MS_DR15_ESg1_1e4 (an MS-associated TCR); (e) MS_DR15_ESg1_Mun1e1 (an MS-associated TCR); Ob. 1A12 (MBP-specific); and NL228_COVID_1 (COVID-specific).

As shown in FIG. 3, 3 out of the 4 MS-associated TCRs tested responded to the ZO-1-encoding constructs. Each construct encodes a 50-mer string harboring the relevant epitopes. Control TCRs behaved as expected and served as an internal control for translation/presentation of the constructs in question.

The data suggests that the ZO-1 antigen is processed and presented, and validates ZO-1 as a candidate target antigen in MS.

### Example 3—Identification of Additional Antigens Bound by MS-Associated TCRs

The three human patient-derived MS-associated TCRs (ESg1_1, ESg1_2 and ESg1_Mun1) were the focus of further deorphanization studies. These three TCRs are highly related at the sequence level, belong to the “PG1/CAISESW” cluster and share the following TCRβ motif: CAISESW [TSA]GG[TSA]DTQYF+TCRBV10-03+TCRBJ02-03.

The further deorphanization efforts identified six additional antigens to which all three MS-associated TCRs bind. These additional antigens are summarized in the table below.

| | | SEQ | | SEQ |
|---|---|---|---|---|
| Peptide | Peptide | ID | | ID |
| Name | Sequence | NO | 9mer | NO |
| Mimotope/ | GGGIALWESHDVGGG | 8 | IALWESHDV | 9 |
| Synthetic | | | | |
| (Syn-2) | | | | |
| PXDC1_ | YGLVAIKEAHDIETR | 10 | VAIKEAHDI | 11 |
| YGL15 | | | | |
| HuTV_ | TPYIGLAESHDNVCD | 12 | IGLAESHDN | 13 |
| TPY15 | | | | |
| ZO-2 | MEELIWEQYTVTLQK | 14 | ELIWEQYTV | 15 |
| ZO-3 | MEELTIWEQHTATLS | 16 | LTIWEQHTA | 17 |
| POLN_ | IPILAVMESHAIQVN | 18 | LAVMESHAI | 19 |
| IPI-15 | | | | |

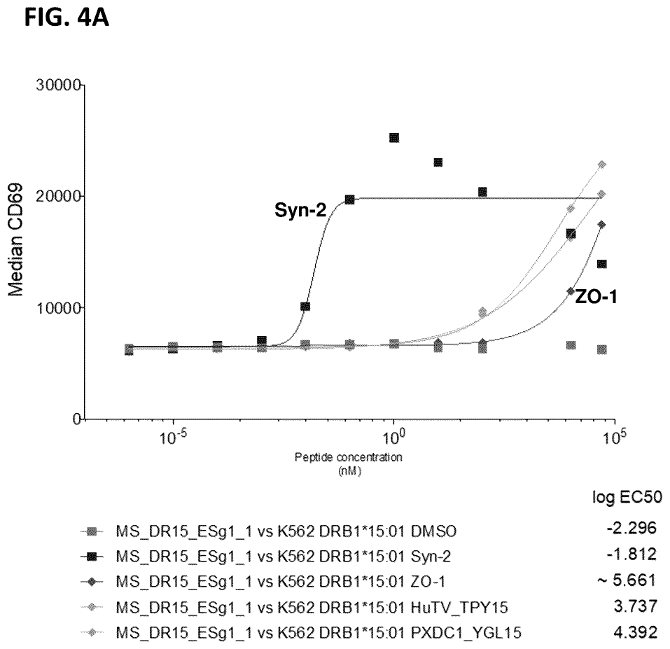

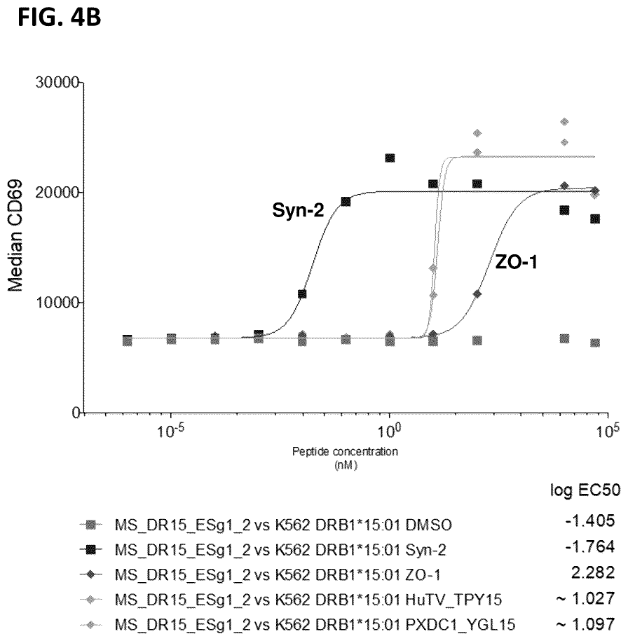

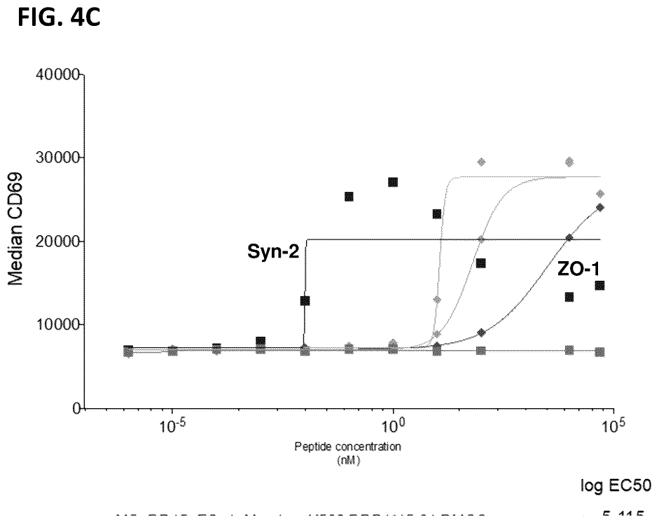

Deorphanization data for peptides PXDC1_YGL15 and HuTV_TPY15 is provided in FIG. 4A-4C for ESg1_1, ESg1_2 and ESg1_Mun1, respectively.

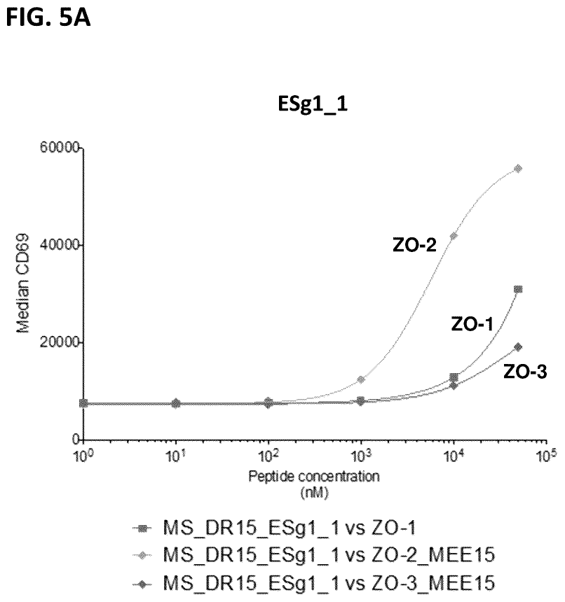

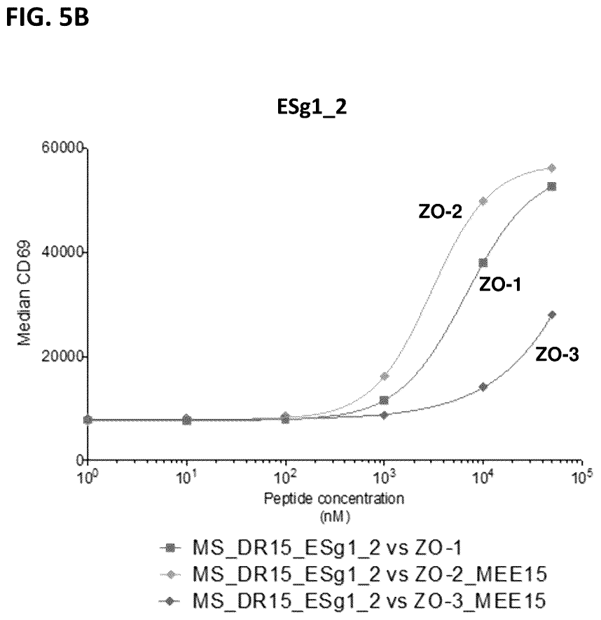

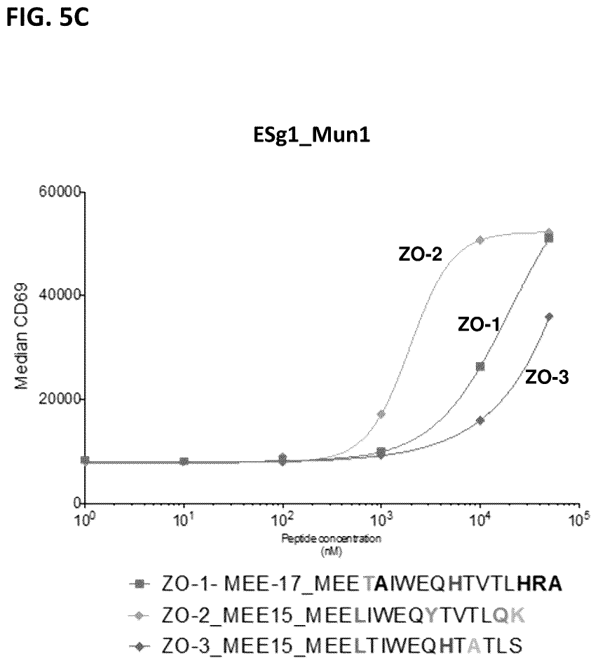

Deorphanization data for the ZO-2 and ZO-3 peptides is shown in FIG. 5A-5C for ESg1_1, ESg1_2 and ESg1_Mun1, respectively. ZO-1 is one of three members of the Zona Occluden (ZO) family of proteins encoded by TJP1, TJP2 and TJP3, respectively. ZO proteins are closely related adaptor proteins that link the tight junction (TJ) transmembrane protein (claudin, occludin, etc.) complexes at the cytoplasmic surface of intercellular junctions to the actin cytoskeleton. ZO-1, ZO-2 and ZO-3 form components of the tight junction barrier at the blood brain barrier, with similar functions and overlapping expression profiles. Based on their sequence similarity, expression profiles and role in BBB function it is hypothesized that similar epitopes derived from ZO-1, ZO-2 and ZO-3 in the context of HLA-DRB1*1501 elicit responses from MS TCRs and provide the rationale to prevent or inhibit anti-Zona Occludens adaptive immune responses.

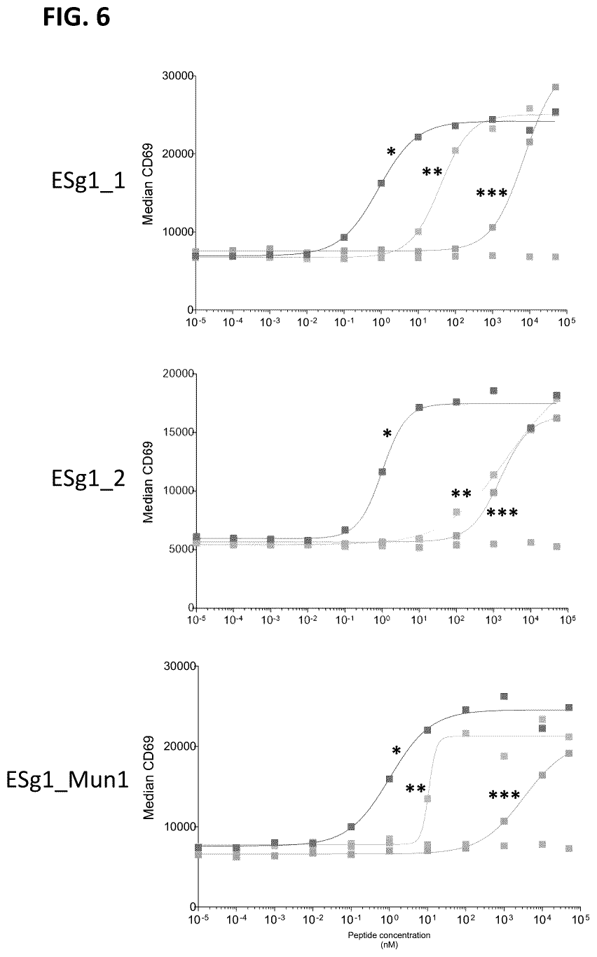

Deorphanization data for peptide POLN_IPI-15 is provided in FIG. 6 for ESg1_1, ESg1_2 and ESg1_Mun1 TCRs.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1-43. (canceled)

44. A soluble peptide-major histocompatibility complex (pMHC), wherein the MHC is HLA-DRB1*15 and the peptide comprises, consists essentially of, or consists of an amino acid sequence chosen from TAIWEQHTV (SEQ ID NO: 7), IALWESHDV (SEQ ID NO:9), VAIKEAHDI (SEQ ID NO: 11), IGLAESHDN (SEQ ID NO: 13), ELIWEQYTV (SEQ ID NO: 15), LTIWEQHTA (SEQ ID NO: 17), and LAVMESHAI (SEQ ID NO: 19).

45. A soluble pMHC multimer comprising two or more of the pMHC of claim 44.

46. The soluble pMHC multimer of claim 45, which is a pMHC dimer, tetramer, or pentamer.

47. The soluble pMHC of claim 44 or multimer thereof, conjugated or fused to a T cell inhibitor.

48. The soluble pMHC of claim 47 or multimer thereof, wherein the T cell inhibitor is an agonist of an immune checkpoint molecule.

49. The soluble pMHC of claim 48 or multimer thereof, wherein the immune checkpoint molecule is chosen from programmed cell death-1 (PD-1), cytotoxic T-lymphocyte-associated antigen 4 (CTLA-4), T cell immunoreceptor with Ig and ITIM domains (TIGIT), T-cell immunoglobulin domain and mucin domain 3 (TIM-3), V-domain Ig suppressor of T cell activation (VISTA), and B and T lymphocyte attenuator (BTLA).

50. The soluble pMHC of claim 47 or multimer thereof, wherein the T cell inhibitor is an anti-CD3 binding agent.

51. The soluble pMHC of claim 44 or multimer thereof, conjugated to an agent.

52. The soluble pMHC of claim 51 or multimer thereof, wherein the agent is chosen from a toxin, a radioisotope, a radiation sensitizing agent, a detectable label, a half-life extending moiety, and any combination thereof.

53. A nanoparticle conjugated to the soluble pMHC of claim 44 or a multimer thereof.

54. A composition comprising the soluble pMHC of claim 44 or a multimer thereof, wherein the composition is suitable for administration to a subject.

55. A method of preventing or inhibiting a multiple sclerosis (MS)-associated adaptive immune response in a subject in need thereof, the method comprising administering to the subject the soluble pMHC of claim 44 or multimer thereof in an amount effective to prevent or inhibit the MS-associated adaptive immune response in the subject.

56. A method of treating multiple sclerosis in a subject in need thereof, the method comprising administering to the subject a therapeutically effective amount of the soluble pMHC of claim 44 or a multimer thereof.

57. A method of preventing or inhibiting a multiple sclerosis (MS)-associated adaptive immune response in a subject in need thereof, the method comprising administering to the subject a therapeutically effective amount of an antibody that specifically binds a TCRBV10 region.

58. The method of claim 57, wherein the antibody is the antigen binding domain of an engineered cell surface receptor, and wherein the method comprises administering a therapeutically effective amount of a population of immune cells expressing the engineered cell surface receptor to the subject.

59. A genetically modified cell that expresses a T cell receptor (TCR) comprising a TCRβ chain comprising a TCRβ CDR3 sequence of one of SEQ ID NOs: 1-3.

60. The cell of claim 59, wherein the TCRβ CDR3 sequence is CAISESWTGGSDTQYF (SEQ ID NO: 1) and the TCRα CDR3 sequence is CIVRPNTGTASKLTF (SEQ ID NO: 4); the TCRβ CDR3 sequence is CAISESWAGGTDTQYF (SEQ ID NO: 2) and the TCRα CDR3 sequence is CIVRGNTGTASKLTF (SEQ ID NO: 5); or the TCRβ CDR3 sequence is CAISEGWTGNTDTQYF (SEQ ID NO: 3) and the TCRα CDR3 sequence is CIVRGNTGTASKLTF (SEQ ID NO: 5).

61. The cell of claim 59, wherein the cell is an immune regulatory cell, optionally wherein the immune regulatory cell is a regulatory T cell (Treg).

62. A method of preventing or inhibiting a multiple sclerosis (MS)-associated adaptive immune response in a subject in need thereof, the method comprising administering to the subject a therapeutically effective amount of immune regulatory cells as defined in claim 61.

63. A nucleic acid adapted to specifically reduce expression by RNA interference (RNAi) of: a TCRβ chain comprising a TCRBV10.3 region; a TCRβ chain comprising a TCRβ CDR3 sequence chosen from CAISESWTGGSDTQYF (SEQ ID NO: 1), CAISESWAGGTDTQYF (SEQ ID NO: 2), CAISEGWTGNTDTQYF (SEQ ID NO: 3), or any combination thereof; or a TCRβ chain comprising a TCRα CDR3 sequence chosen from CIVRPNTGTASKLTF (SEQ ID NO: 4), CIVRGNTGTASKLTF (SEQ ID NO: 5), or both.
